(19) United States
(12) Patent Application Publication
(10) Pub. No.: US 2016/0032301 A1
(43) Pub. Date: Feb. 4, 2016

(54) GUARD CELL EXPRESSION CASSETTES COMPOSITIONS AND METHODS OF USE THEREOF

(71) Applicant: SYNGENTA PARTICIPATIONS AG, Basel (CH)

(72) Inventor: Michael L. NUCCIO, Research Triangle Park, NC (US)

(73) Assignee: SYNGENTA PARTICIPATIONS AG, Basel (CH)

(21) Appl. No.: 14/776,764

(22) PCT Filed: Mar. 6, 2014
(86) PCT No.: PCT/US14/21085

§ 371 Date: Sep. 15, 2015

Related U.S. Application Data
(60) Provisional application No. 61/787,781, filed on Mar. 15, 2013.

(51) Int. Cl. C12N 15/82; C12N 9/24; C12N 15/74

(52) U.S. Cl. CPC C12N 15/8223; C12N 15/743; C12N 9/2402; C12N 15/8261; C12Y 302/01031

(57) ABSTRACT

The invention is directed to artificial transcription regulating polynucleotides that confer guard cell regulated expression and methods of use thereof. The present invention further provides methods of using the transcription regulating polynucleotides and plants and plant parts thereof comprising the transcription regulating polynucleotides. In agricultural biotechnology, plants can be modified according to one's needs. One way to accomplish this is by using modern genetic engineering techniques. For example, by introducing a gene of interest into a plant, the plant can be specifically modified to express a desirable phenotypic trait.

### FIELD OF THE INVENTION

The present invention relates to the fields of plant functional genomics, molecular biology, genetic engineering and selective regulation of gene expression in plants. In particular, the present invention describes artificial transcription regulating polynucleotides capable of conferring guard cell regulated expression.

### STATEMENT REGARDING ELECTRONIC FILING OF A SEQUENCE LISTING

A Sequence Listing in ASCII text format, submitted under 37 C.F.R. §1,821, entitled 73701-WO-REG-ORG-P-1_Sequence_Listing_ST25, 55.1 KB bytes in size, generated on Feb. 18, 2014 and filed via EFS-Web, is provided in lieu of a paper copy. This Sequence Listing is hereby incorporated herein by reference into the specification for its disclosures.

### BACKGROUND OF THE INVENTION

In agricultural biotechnology, plants can be modified according to one's needs. One way to accomplish this is by using modern genetic engineering techniques. For example, by introducing a gene of interest into a plant, the plant can be specifically modified to express a desirable phenotypic trait. For this, plants are transformed most commonly with a heterologous gene comprising a promoter region, a coding region and a termination region. When genetically engineering a heterologous gene for expression in plants, the selection of a promoter is often a factor. While it can be desirable to express certain genes constitutively, i.e. throughout the plant at all times and in most tissues and organs, other genes are more desirably expressed only in response to particular stimuli or confined to specific cells or tissues.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters”, if the promoters direct RNA synthesis preferentially in certain tissues (RNA synthesis can occur in other tissues at reduced levels). Since patterns of expression of a nucleic acid of interest introduced into a plant, plant tissue or plant cell are controlled using promoters, there is an ongoing interest in the isolation of novel promoters that are capable of controlling the expression of a nucleic acid of interest at certain levels in specific tissue types or at specific plant developmental stages.

Stomatal pores in the epidermis of plant leaves enable the control of plant water loss and gas exchange including the influx of carbon dioxide into plants from the atmosphere. Carbon dioxide is taken up for photosynthetic carbon fixation and water is lost through the process of transpiration though the stomatal pores. Each stomata consists of a specialized pair of guard cells, which can control the size of the stomatal pore opening by modulating cellular turgor pressure. Water use efficiency of plants is an important aspect of plant biotechnological applications and agriculture. Water use efficiency defines how well a plant can balance water loss through the stomata with the net CO₂ uptake into leaves for photosynthesis resulting in biomass accumulation. Several biotic and abiotic factors influence stomatal aperture thereby regulating water use of a plant in a given condition. For example, the concentration of CO₂ regulates stomatal aperture in that high levels of CO₂ reduce stomatal aperture and low levels of CO₂ increase stomatal aperture. Thus external atmospheric CO₂ exerts some control over CO₂ influx into plants and plant transpiration.

The number of guard cell-specific or guard cell-preferred promoters in the art is very limited (EP-A1 1111 051; Plesch 2001). It is advantageous to have the choice of a variety of different promoters so that the most suitable promoter may be selected for a particular gene, construct, cell, tissue, plant or environment. Moreover, the increasing interest in transforming plants with multiple plant transcription units and the potential problems associated with using common regulatory sequences for these purposes merit having a variety of promoter sequences available. Regulatory elements that are able to control specific expression of heterologous genes of interest in guard cells could have significant use plant biotechnology and agriculture. For instance genes could be expressed to more tightly regulate the aperture of stomatal pores to improve water use in plants.

### SUMMARY OF THE INVENTION

The present invention provides compositions and methods for modulating gene expression in plant guard cells. Accordingly, in one aspect, the present invention provides a recombinant polynucleotide comprising, consisting essentially of, or consisting of a nucleic acid selected from the group consisting of (a) the nucleic acid of SEQ ID NO:1; and (b) a nucleic acid that is at least 95% identical to the nucleic acid of (a). Alternatively, the present invention provides a recombinant polynucleotide comprising a nucleic acid having at least 90% identity over the entire length of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28. In addition, the present invention provides a recombinant polynucleotide comprising a nucleic acid selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 and SEQ ID NO:28.

In some aspects, the recombinant polynucleotide of the invention (e.g., SEQ ID NO:1) can be operably linked to at least one polynucleotide of interest. In other aspects, the recombinant polynucleotide of the invention (e.g., SEQ ID NO:1) can be operably linked to at least one intron. In still other aspects, the recombinant polynucleotide of the invention can be operably linked to at least one intron and at least one exon. In still further aspects, the recombinant polynucleotide of the invention that is operably linked to at least one intron or to at least one intron and at least one exon, can be optionally, further operably linked to at least one enhancer such as a TMV omega translational enhancer and/or a Kozak sequence. In other aspects, the recombinant polynucleotide of the invention operably linked to at least one intron or to at least one intron and at least one exon, and optionally further linked to a TMV omega translational enhancer and/or a Kozak sequence can be further operably linked to at least one polynucleotide of interest.

In an additional aspect, the present invention provides an expression cassette comprising, consisting essentially of, or consisting of a recombinant polynucleotide of the invention. In a further aspect, the expression cassette of the invention can comprise, consist essentially of, or consist of a recombinant polynucleotide of the invention operably linked to at least one polynucleotide of interest. In still other aspects, the present invention provides an expression cassette comprising, consisting essentially of, or consisting of a recombinant polynucleotide of the invention operably linked to at least one intron or to at least one intron and at least one exon. In still further aspects, the present invention provides an expression cassette comprising, consisting essentially of, or consisting of the recombination polynucleotide of the invention operably linked to at least one intron or to at least one intron and at least one exon, which can be optionally further operably linked to one or more enhancers. Optionally the enhancer may be a TMV omega translational enhancer and/or a Kozak sequence. In other aspects, the present invention provides an expression cassette comprising, consisting essentially of, or consisting of a recombinant polynucleotide of the invention operably linked to at least one intron or to at least one intron and at least one exon, and optionally further operably linked to a TMV omega translational enhancer and/or a Kozak sequence that is further operably linked to at least one polynucleotide of interest. A polynucleotide of interest may include an abscisic acid receptor or a modified abscisic acid receptor.

In a further aspect, the present invention provides an expression cassette comprising a recombinant polynucleotide that is at least about 95% identical to the nucleic acid of SEQ ID NO:1. The present invention also provides an expression cassette comprising a recombinant polynucleotide that is at least about 90% identical to the nucleic acid of SEQ ID NO:1; SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28.

In a still further aspect, the present invention provides a cell, a plant or plant part comprising, consisting essentially of, or consisting of a recombinant nucleotide of the invention or expression cassette of the invention.

In an additional aspect, the present invention provides a method of expressing a polynucleotide of interest in a guard cell of a plant, comprising introducing into a plant cell a recombinant polynucleotide of the invention and/or an expression cassette of the invention, regenerating the plant cell into a plant stably transformed with said recombinant polynucleotide and/or said expression cassette of the invention, wherein the polynucleotide of interest is expressed in the guard cells of said stably transformed plant.

In a further aspect, a method of modulating guard cell function (e.g., stomata opening and closing) is provided, the method comprising introducing into a plant cell a recombinant polynucleotide of the invention and/or an expression cassette of the invention, regenerating the plant cell into a plant stably transformed with said recombinant polynucleotide and/or said expression cassette of the invention, thereby modulating the function of the guard cells of the stably transformed plant.

A further aspect of the invention provides a method of improving plant response to water deficit and plant water use efficiency, comprising introducing into a plant cell a recombinant polynucleotide of the invention and/or an expression cassette of the invention, regenerating the plant cell into a plant stably transformed with said recombinant polynucleotide and/or said expression cassette of the invention, thereby improving response to water deficit and water use efficiency in the stably transformed plant.

In some aspects, the present invention provides a method of modulating photoassimilation rate, comprising introducing into a plant cell a recombinant polynucleotide of the invention and/or an expression cassette of the invention, regenerating the plant cell into a plant stably transformed with said recombinant polynucleotide and/or said expression cassette of the invention, thereby modulating the photoassimilation rate in the stably transformed plant.

In an additional aspect, a method of modulating the rate of plant transpiration is provided, comprising introducing into a plant cell a recombinant polynucleotide of the invention and/or an expression cassette of the invention, regenerating the plant cell into a plant stably transformed with said recombinant polynucleotide and/or said expression cassette of the invention, thereby modulating the rate of transpiration in the stably transformed plant.

In other aspects, a method of producing a plant having modulated guard cell function is provided, the method comprising introducing into a plant cell the recombinant polynucleotide of and/or the expression cassette of the invention, regenerating the plant cell into a plant and/or plant part stably transformed with said recombinant polynucleotide and/or said expression cassette, thereby producing a stably transformed plant having modulated guard cell function.

Additionally provided are plants, plant parts, plant cells comprising a recombinant polynucleotide of the invention and/or an expression cassette of the invention as well as crops and products produced therefrom. In some particular aspects, the invention provides seeds and progeny plants produced from the plants of the invention.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The present invention provides compositions and methods for altering gene expression in plant guard cells, thereby providing the ability, for example, to manipulate the exchange of water and/or carbon dioxide (CO₂) through plant stomata (e.g., modify net CO₂ uptake and water use efficiency and/or activity of CO₂ sensor genes such as the genes described in WO08134571 herein incorporated by reference) and modulate photosynthetic assimilation rate and/or water loss through the process of transpiration.

The present invention is directed to recombinant polynucleotides and nucleic acid constructs (e.g., expression cassettes) useful for the expression of nucleic acids of interest in a guard cell preferred or guard cell specific pattern (e.g., transcription regulating polynucleotides). Thus, a nucleic acid construct (e.g., expression cassette) of the present invention comprises at least an artificial transcription regulating polynucleotide encoded by the nucleic acid of SEQ ID NO:1.

Accordingly, in one embodiment, the present invention provides a recombinant polynucleotide comprising, consisting essentially of or consisting of a nucleic acid selected from the group consisting of (a) the nucleic acid of SEQ ID NO:1; (b) a nucleic acid that is at least about 95% identical to the nucleic acid of (a); (c) a nucleic acid that differs from the nucleic acid of (a) or (b) due to the degeneracy of the genetic code; and (d) any combination of (a), (b) and (c).

In another embodiment, the present invention provides a recombinant polynucleotide comprising, consisting essentially of or consisting of a nucleic acid selected from the group consisting of (a) the nucleic acid of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28 and any combination thereof; (b) a nucleic acid that is at least about 75% identical to the nucleic acid of (a); (c) a nucleic acid that differs from the nucleic acid of (a) or (b) due to the degeneracy of the genetic code; and (d) any combination of (a), (b) and (c). The recombinant polynucleotides identified in SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 and SEQ ID NO:28 contain SEQ ID NO: 1. SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 and SEQ ID NO:28 provide SEQ ID NO: 1 in combination with various other nucleic acid elements including, but not limited to, introns, exons, and translational enhancers.

Thus, in some embodiments, a nucleic acid of the present invention can be a nucleic acid having substantial identity (e.g., at least about 70% to about 100% identity) to a nucleic acid of SEQ ID NO:1, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28 or any combination thereof. Accordingly, in some embodiments, a nucleic acid of the invention that is substantially identical to a nucleic acid of SEQ ID NO:1, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28 has an identity of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, and the like, or any range therein, to the respective nucleic acid (e.g., SEQ ID NOs:1 or SEQ ID NOs:10-28). In particular embodiments, a nucleic acid of the invention has at least about 80% identity to the nucleic acid of SEQ ID NOs:1 or SEQ ID NOs:10-28, at least about 85% identity to the nucleic acid of SEQ ID NOs:1 or SEQ ID NOs:10-28, at least about 90% identity to the nucleic acid of SEQ ID NOs:1 or SEQ ID NOs:10-28, at least about 95% identity to a nucleic acid of SEQ ID NOs:1 or SEQ ID NOs:10-28.

In some embodiments of the invention, the recombinant polynucleotide can be operably linked to a polynucleotide of interest. In other embodiments, the recombinant polynucleotide can be comprised in an expression cassette. In further embodiments, when operably linked to a polynucleotide of interest, the recombinant polynucleotide of the invention can confer specific or preferred expression of said polynucleotide of interest in guard cells of a plant or plant part transformed with said recombinant polynucleotide or said expression cassette comprising said recombinant polynucleotide. The polynucleotide of interest may comprise a transgene which provides herbicide resistance, fungal resistance, insect resistance, resistance to disease, resistance to nematodes, male sterility, resistance to abiotic stress or which alters the oil profiles, the fatty acid profiles, the amino acids profiles or other nutritional qualities of the seed. Of particular interest are transgenes involved in resistance to abiotic stress. For example, transgenes involved in the abscisic acid (ABA) response pathway, such as, ABA receptors, PP2Cs or SnRK1 are possible transgenes of interest. ABA receptors may be modified to be constitutively active or to recognize molecules other than ABA. Please see, WO 2010/093954; WO2011/139798 (modified ABA receptors); WO2013006263 (constitutively active ABA receptors), all of which are hereby incorporated by reference.

As would be well understood by those of skill in the art, an expression cassette comprising the nucleic acid of SEQ ID NO:1 can further comprise a Kozak sequence as well as other nucleotide sequences useful for construction of expression cassettes including, but not limited to, restriction endonuclease recognition sites. Both Kozak sequences and restriction endonuclease sites are well known in the art (see, e.g., M. Kozak, Nucleic Acids Res. 15 (20): 8125-8148 (1987); Nakagawa et al. Nucleic Acids Res. 36(3): 861-871 (2008); Sambrook (Molecular Cloning: A Laboratory Manual (2^{nd} ed., Cold Spring Harbor Laboratory Press, Plainview, N.Y. (1989)). Thus, in one embodiment, an expression cassette of this invention can comprise the nucleic acid of SEQ ID NO:1 operably linked at the 3′ end to a Kozak sequence. In another embodiment, an expression cassette of this invention comprising the nucleic acid of SEQ ID NO:1, can further comprise a restriction endonuclease recognition site at the 5′ and/or 3′ end of said expression cassette. In further embodiments, an expression cassette of this invention comprising the nucleic acid of SEQ ID NO:1 operably linked at the 3′ end to a Kozak sequence, can further comprise a restriction endonuclease recognition site linked at the 5′ and/or 3′ end to the expression cassette.

Thus, in a representative embodiment of the invention, an expression cassette can comprise the nucleic acid of SEQ ID NO: 26 which comprises in the following order, 5′ to 3′, the nucleic acid of SEQ ID NO:1 and a Kozak sequence.

In further embodiments of this invention, an expression cassette of this invention can comprise the nucleic acid of SEQ ID NO:1 operably linked at the 3′ end to an intron. Any intron useful with this invention can be used including, but not limited to, an intron from any guard cell gene and/or any ubiquitin gene. In still other embodiments, an intron useful with this invention can be an intron as identified in the Maize Genome Database (maizegdb.org) including, but not limited to, GRMZM2G061447, GRMZM2G019200, GRMZM2G098237, GRMZM2G120596 and/or GRMZM2G132854. In some representative embodiments, the intron can be encoded by the nucleic acid of SEQ ID NO:2, SEQ ID NO:3, SEQ ID NO:4, SEQ ID NO:5, SEQ ID NO:6, SEQ ID NO:7, and/or SEQ ID NO:8. In a particular embodiment, the intron can be encoded by the nucleic acid of SEQ ID NO: 2. In further embodiment, the intron can be encoded by the nucleic acid of SEQ ID NO:3. In some embodiments, an expression cassette comprising the nucleic acid of SEQ ID NO:1 operably linked at the 3′ end to an intron can further optionally comprise a Kozak sequence operably linked to the 3′ end of said intron.

Accordingly, in some particular embodiments, an expression cassette comprising the nucleic acid of SEQ ID NO:1 can comprise the nucleic acid of SEQ ID NO:2, SEQ ID NO:3, SEQ ID NO:4, SEQ ID NO:5, SEQ ID NO:6, SEQ ID NO:7, and/or SEQ ID NO:8 operably linked to the 3′ end of the nucleic acid of SEQ ID NO:1. As would be well understood by those of skill in the art, an expression cassette comprising the nucleic acid of SEQ ID NO:2, SEQ ID NO:3, SEQ ID NO:4, SEQ ID NO:5, SEQ ID NO:6, SEQ ID NO:7, and/or SEQ ID NO:8 operably linked to the 3′ end of the nucleic acid of SEQ ID NO:1 can further comprise a Kozak sequence operably linked to the 3′ end of the nucleic acid of SEQ ID NO:2, SEQ ID NO:3, SEQ ID NO:4, SEQ ID NO:5, SEQ ID NO:6, SEQ ID NO:7, and/or SEQ ID NO:8.

In still further embodiments, an expression cassette of the invention can comprise the nucleic acid of SEQ ID NO:1 operably linked at the 3′ end to an intron, wherein the intron is further operably linked at its 3′ end to an exon or portion thereof. The exon or portion thereof can be any exon or portion thereof that is useful with the invention. In representative embodiments, the exon or portion thereof operably linked to the intron can be encoded by the nucleic acid of SEQ ID NO:9.

As would be understood by those of skill in the art, the exon or portion thereof comprised in an expression cassette of the invention can be further optionally linked at the 3′ end to a Kozak sequence. Thus in some embodiments, an expression cassette comprising the nucleic acid of SEQ ID NO:1 operably linked at the 3′ end to an intron, which intron can be operably linked at the 3′ end to an exon or portion thereof, can further optionally comprise a Kozak sequence operably linked to the 3′ end of the exon. Thus, in a representative embodiment, an expression cassette of the invention comprises the nucleic acid of SEQ ID NO:25 and/or SEQ ID NO:27.

In some particular embodiments, an expression cassette comprising SEQ ID NO:1 operably linked to an intron at its 3′ end, wherein the intron is further operably linked at its 3′ end to an exon or portion thereof can comprise the nucleic acid of SEQ ID NO:17, SEQ ID NO:18, SEQ ID NO:19, SEQ ID NO:20, SEQ ID NO:21, SEQ ID NO:22, and/or SEQ ID NO:23. As would be well understood by those of skill in the art, the nucleic acid of SEQ ID NO:17, SEQ ID NO:18, SEQ ID NO:19, SEQ ID NO:20, SEQ ID NO:21, SEQ ID NO:22, and/or SEQ ID NO:23 can further comprise a Kozak sequence operably linked to the 3′ end of said nucleic acid.

In a representative embodiment of the invention, an expression cassette can comprise the nucleic acid of SEQ ID NO: 25 and/or SEQ ID NO: 27, each of which comprise in the following order, 5′ to 3′, the nucleic acid of SEQ ID NO:1, an intron, a portion of an exon and a Kozak sequence.

In still further embodiments, an expression cassette comprising the nucleic acid of SEQ ID NO:1 operably linked at the 3′ end to an intron, which intron is operably linked at the 3′ end to an exon or portion thereof, can further optionally comprise a translational enhancer, e.g., TMV omega translational enhancer, operably linked to the 3′ end of the exon or portion thereof or to the 3′ end of a Kozak sequence operably linked to the 3′ end of the exon or portion thereof. TMV omega translational enhancer is known in the art (see, e.g., Gallie et al., Nucleic Acids Res. 20:4631-4638 (1992); Gallie D R Nucleic Acids Res. 30:3401-3411 (2002)) and Pfeiffer et al. Proc. Natl. Acad Sci 109(17):6626-6631 (2012)). In a representative embodiment, a TMV omega translational enhancer can be encoded by the nucleic acid of SEQ ID NO:29.

Accordingly, in some embodiments, an expression cassette is provided comprising the nucleic acid of SEQ ID NO:1 operably linked at the 3′ end to an intron, which intron is operably linked at the 3′ end to an exon, can further optionally comprise both a TMV omega translational enhancer and a Kozak sequence, wherein the Kozak sequence can be operably linked to the 3′ end of the exon and the TMV omega translational enhancer can be operably linked to the 3′ end of the Kozak sequence. Thus, in representative embodiments, an expression cassette of the invention can comprise in the following order, 5′ to 3′, the nucleic acid of SEQ ID NO:1, an intron, an exon, and optionally, a Kozak sequence and/or a TMV omega translational enhancer.

Further, in some particular embodiments, an expression cassette of this invention can comprise the nucleic acid of SEQ ID NO:24, which comprises in the following order, 5′ to 3′, the nucleic acid of SEQ ID NO:1, an intron, a portion of an exon and the TMV omega translational enhancer sequence.

As would be well understood by those of skill in the art, an expression cassette of this invention can optionally further comprise a restriction endonuclease recognition site linked to the 5′ and/or 3′ end of said expression cassette. Thus, in some embodiments of this invention, an expression cassette can comprise in the following order, 5′ to 3′, a restriction endonuclease recognition site, the nucleic acid of SEQ ID NO:1, and a restriction endonuclease recognition site. In other embodiments, an expression cassette of the invention can comprise in the following order, 5′ to 3′, a restriction endonuclease recognition site, the nucleic acid of SEQ ID NO:1, a Kozak sequence, and a restriction endonuclease recognition site. In still other embodiments, an expression cassette of the invention can comprise in the following order, 5′ to 3′, a restriction endonuclease recognition site, the nucleic acid of SEQ ID NO:1, an intron, and a restriction endonuclease recognition site. In a further embodiment, an expression cassette of the invention can comprise in the following order, 5′ to 3′, a restriction endonuclease recognition site, the nucleic acid of SEQ ID NO:1, an intron, a Kozak sequence, and a restriction endonuclease recognition site. In additional embodiments of the invention, an expression cassette can comprise in the following order, 5′ to 3′, a restriction endonuclease recognition site, the nucleic acid of SEQ ID NO:1, an intron, an exon, and a restriction endonuclease recognition site. In additional embodiments of the invention, an expression cassette can comprise in the following order, 5′ to 3′, a restriction endonuclease recognition site, the nucleic acid of SEQ ID NO:1, an intron, an exon, a Kozak sequence, and a restriction endonuclease recognition site. In further embodiments of the invention, an expression cassette can comprise in the following order, 5′ to 3′, a restriction endonuclease recognition site, the nucleic acid of SEQ ID NO:1, an intron, an exon, a TMV omega translational enhancer, and a restriction endonuclease recognition site. In still further embodiments of the invention, an expression cassette can comprise in the following order, 5′ to 3′, a restriction endonuclease recognition site, the nucleic acid of SEQ ID NO:1, an intron, an exon, a TMV omega translational enhancer, a Kozak sequence, and a restriction endonuclease recognition site.

As used herein, the terms “nucleic acid,” “nucleic acid molecule,” “nucleotide sequence” and “polynucleotide” can be used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the polynucleotides and polypeptide sequences of this invention. “Orthologous,” as used herein, refers to homologous nucleotide sequences and/or amino acid sequences in different species that arose from a common ancestral gene during speciation. A homologue of this invention has a substantial sequence identity (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to the nucleic acids of the invention.

A “heterologous” nucleic acid is a nucleic acid not naturally associated with a host cell into which it is introduced, including non-naturally occurring multiple copies of a naturally occurring nucleic acid.

As used herein, the term “chimeric” indicates that a DNA sequence, such as a vector or a gene, is comprised of two or more DNA sequences of distinct origin that are fused together by recombinant DNA techniques resulting in a DNA sequence, which does not occur naturally.

A “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleic acid naturally associated with a host cell into which it is introduced.

In some embodiments, the recombinant nucleic acids molecules, polynucleotide sequences and polypeptides of the invention are “isolated.” An “isolated” nucleic acid molecule, an “isolated” nucleotide sequence or an “isolated” polypeptide is a nucleic acid molecule, nucleotide sequence or polypeptide that, by the hand of man, exists apart from its native environment and is therefore not a product of nature. An isolated nucleic acid molecule, nucleotide sequence or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

In other embodiments, an isolated nucleic acid molecule, nucleotide sequence or polypeptide may exist in a non-native environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the recombinant nucleic acid molecules, nucleotide sequences and their encoded polypeptides are “isolated” in that, by the hand of man, they exist apart from their native environment and therefore are not products of nature, however, in some embodiments, they can be introduced into and exist in a recombinant host cell. “Artificial polynucleotides or polypeptides”, “engineered polynucleotides or polypeptides”, “designed polynucleotides or polypeptides”, “synthetic polynucleotides or polypeptides”, “non-naturally occurring polynucleotides or polypeptides” or the like were created by human intervention and are not wild type polynucleotides or polypeptides.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter or transcription regulating polynucleotide is operably associated with a nucleotide sequence if the promoter or transcription regulating polynucleotide effects the transcription or expression of said nucleotide sequence. Those skilled in the art will appreciate that a control sequences (e.g., promoter or transcription regulating polynucleotide) need not be contiguous with a nucleotide sequence to which it is operably associated, as long as the control sequence(s) function to direct the expression thereof. Thus, for example, intervening untranslated, yet transcribed, sequences can be present between a promoter or transcription regulating polynucleotide and a nucleotide sequence to be expressed, and the promoter or transcription regulating polynucleotide can still be considered “operably linked” to the nucleotide sequence to be expressed.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 70%, at least about 75%, at least about 80%, least about 85%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 150 residues in length. Thus, in some embodiments of the invention, the substantial identity exists over a region of the sequences that is at least about 50, about 60, about 70, about 80, about 90, about 100, about 110, about 120, about 130, about 140, about 150, or more residues in length. In some particular embodiments, the sequences are substantially identical over at least about 150 residues. In a further embodiment, the sequences are substantially identical over the entire length of the coding regions or reference sequence. For example, a polynucleotide of the invention could have 80%, 85%, 90%, 95%, 96%, 97%, 98%, 99% or 100% identity over the entire length of SEQ ID NO: 1. Furthermore, in representative embodiments, substantially identical nucleotide or protein sequences perform substantially the same function (e.g., conferring guard cell specific and/or guard cell preferred expression).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially identical when the two sequences hybridize to each other under stringent conditions. In some representative embodiments, two nucleotide sequences considered to be substantially identical hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

In particular embodiments, a further indication that two nucleotide sequences or two polypeptide sequences are substantially identical can be that the protein encoded by the first nucleic acid is immunologically cross reactive with, or specifically binds to, the protein encoded by the second nucleic acid. Thus, in some embodiments, a polypeptide can be substantially identical to a second polypeptide, for example, where the two polypeptides differ only by conservative substitutions.

As used herein, the terms “express,” “expresses,” “expressed” or “expression,” and the like, with respect to a nucleotide sequence (e.g., RNA or DNA) indicates that the nucleotide sequence is transcribed and, optionally, translated. Thus, a nucleotide sequence may express a polypeptide of interest or a functional untranslated RNA. A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers, and the like.

“Expression cassette” as used herein means a nucleic acid sequence capable of directing expression of a particular nucleotide sequence in an appropriate host cell, comprising a transcription regulating polynucleotide operably linked to a polynucleotide of interest, which is operably linked to termination signals. It can also comprise sequences required for proper translation of the nucleotide sequence. The coding region can code for a protein of interest but may also code for a functional RNA of interest, for example antisense RNA or a nontranslated RNA, in the sense or antisense direction. In some embodiments, the expression cassette comprising the nucleotide sequence of interest may be chimeric, meaning that at least one of its components is heterologous with respect to at least one of its other components.

As used herein, “regulatory sequence(s)” means nucleotide sequence(s) located upstream (5′ non-coding sequences), within or downstream (3′ non-coding sequences) of a coding sequence, which influence the transcription, RNA processing or stability, or translation of the associated coding sequence. Regulatory sequences include, but are not limited to, promoters, enhancers, exons, introns, translation leader sequences, termination signals, and polyadenylation signal sequences. Regulatory sequences include natural and synthetic sequences as well as sequences that can be a combination of synthetic and natural sequences. “An artificial regulatory sequence”, “an engineered regulatory sequence”, “a designed regulatory sequence”, “a synthetic regulatory sequence”, “a non-naturally occurring regulatory sequence” is a regulatory sequence created by human intervention and are not wild type regulatory sequences. For example, a wild type regulatory sequence may be altered or designed to improve transcription, translation or expression of a gene. Alternatively, the regulatory sequence may be created without reference to a particular wild type regulatory sequence.

An “enhancer” is a nucleic acid that improves the expression of a polynucleotide or polypeptide. Enhancers may be transcriptional enhancers or translational enhancers. A “transcriptional enhancer” is a nucleic acid that can stimulate promoter activity and can be an innate element of the promoter or a heterologous element inserted to enhance the level or tissue specificity of a promoter. The primary sequence can be present on either strand of a double-stranded DNA molecule, and is capable of functioning even when placed either upstream or downstream from the promoter. The meaning of the term “promoter” can include “promoter regulatory sequences.” The term “translational enhancer sequence” refers to that DNA sequence portion of a gene between the promoter and coding sequence that is transcribed into RNA and is present in the fully processed mRNA upstream (5′) of the translation start codon. The translational enhancer sequence may affect processing of the primary transcript to mRNA, mRNA stability or translation efficiency. Those skilled in the art will be aware of terminator and enhancer sequences that may be suitable for use in performing the invention. Such sequences would be known or may readily be obtained by a person skilled in the art.

In some embodiments, regulatory sequences or regions can be wild type/analogous to the host cell and/or the regulatory sequences can be wild type/analogous to the other regulatory sequences. Alternatively, the regulatory sequences may be heterologous to the host cell and/or to each other (i.e., the regulatory sequences).

“Transcription regulating polynucleotide” refers to a polynucleotide, which lies upstream of the transcription start site and controls the expression of a coding sequence by providing the recognition for RNA polymerase and other factors required for proper transcription. Types of promoters can include, for example, promoters that are constitutive, inducible, temporally regulated, developmentally activated, chemically activated, tissue-preferred and/or tissue-specific. Thus, for example, the nucleic acids of SEQ ID NOs:1 and/or SEQ ID NOs:10-28, as described herein can function as transcription regulating polynucleotides, conferring guard cell specific or preferred expression on a polynucleotide of interest upon a plant comprising said nucleic acids.

“Artificial transcription regulating polynucleotides”; “engineered transcription regulating polynucleotides”; “designed transcription regulating polynucleotides”; “synthetic transcription regulating polynucleotides” or “non-naturally occurring transcription regulating polynucleotides” are changed or created by human intervention and are not wild type. For example, the artificial transcription regulating polynucleotide may be designed without any reference to a specific naturally occurring transcription regulatory polynucleotide or the artificial transcription regulating polynucleotide may modify an existing transcription regulating polynucleotide.

“Regulated promoter” refers to promoters that direct gene expression not constitutively, but in a temporally- and/or spatially-regulated manner, and include both tissue-specific, tissue-preferred, and inducible promoters. It includes natural and synthetic sequences as well as sequences which may be a combination of synthetic and natural sequences. Different promoters may direct the expression of a gene in different tissues or cell types, or at different stages of development, or in response to different environmental conditions.

“Tissue-specific promoter” refers to regulated promoters that are not expressed in all plant cells but only in one or more cell types in specific organs (such as leaves, roots or seeds), specific tissues (e.g., vascular, dermal, parenchymal), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence. “Tissue-preferred promoters”, are promoters that direct RNA synthesis preferentially in certain tissues (i.e., RNA synthesis can occur in other tissues at reduced levels).

As used herein “guard cell” refers to the specialized epidermal cells that regulate the aperture (i.e. opening and closing) of stomata and by this controls the bulk of gas exchange as well as transpiration. These cells are characterized by their highly regulated turgor (i.e. pressure-dependent shape), which causes the stomata to close or to open at states of low or high turgor, respectively. Guard cells derive from epidermal cells and differ from their surrounding epidermal cells not only by their bean like shape but also by their ability to photosynthesize.

“Guard cell-specific transcription” in the context of this invention refers to the transcription of a nucleic acid sequence by a transcription regulating element in a way that the transcription of said nucleic acid sequence in guard-cells contribute to more than 90%, preferably more than 95%, more preferably more than 99% of the entire quantity of the RNA transcribed from said nucleic acid sequence in the entire plant during any of its developmental stage.

“Guard cell-preferential transcription” herein refers to the transcription of a nucleic acid sequence by a transcription regulating element in a way that transcription of said nucleic acid sequence in guard-cells contribute to more than 50%, preferably more than 70%, more preferably more than 80% of the entire quantity of the RNA transcribed from said nucleic acid sequence in the entire plant during any of its developmental stages.

Preferably a transcription regulating polynucleotide of the invention comprises at least one promoter sequence localized upstream of the transcription start of a polynucleotide of interest (e.g., a nucleotide sequence for which transcription is desired) and is capable of inducing transcription of downstream sequences. The transcription regulating polynucleotide may further comprise other elements such as the 5′-untranslated sequences, enhancer sequences, introns, and/or exons.

Promoters can comprise several regions that play a role in function of the promoter. Some of these regions are modular, in other words they can be used in isolation to confer promoter activity or they can be assembled with other elements to construct new promoters. The first of these promoter regions lies immediately upstream of the coding sequence and forms the “core promoter region” often containing consensus sequences, normally 20-70 base pairs immediately upstream of the coding sequence. The core promoter region typically contains a TATA box and often an initiator element as well as the initiation site. Such a region is normally present, with some variation, in most promoters. The core promoter region is often referred to as a minimal promoter region because it is functional on its own to promote a basal level of transcription.

The presence of the core promoter region defines a sequence as being a promoter: if the region is absent, the promoter is non-functional. The core region acts to attract the general transcription machinery to the promoter for transcription initiation. However, the core promoter region is typically not sufficient to provide promoter activity at a desired level. A series of regulatory sequences, often upstream of the core, constitute the remainder of the promoter. The regulatory sequences can determine expression level, the spatial and temporal pattern of expression and, for a subset of promoters, expression under inductive conditions (regulation by external factors such as light, temperature, chemicals and hormones). Regulatory sequences can be short regions of DNA sequence 6-100 base pairs that define the binding sites for trans-acting factors, such as transcription factors. Regulatory sequences can also be enhancers, longer regions of DNA sequence that can act from a distance from the core promoter region, sometimes over several kilobases from the core region. Regulatory sequence activity can be influenced by trans-acting factors including but not limited to general transcription machinery, transcription factors and chromatin assembly factors.

In a representative embodiment, a minimal transcription regulating polynucleotide of this invention can be the nucleic acid of SEQ ID NO:1. In a further embodiment, a minimal transcription regulating polynucleotide of this invention can be a nucleic acid of SEQ ID NOs:10-28.

“Intron” refers to an intervening section of DNA which occurs almost exclusively within a eukaryotic gene, but which is not translated to amino acid sequences in the gene product. The introns are removed from the pre-mature mRNA through a process called splicing, which leaves the exons unchanged, to form an mRNA. For purposes of the present invention, the definition of the term “intron” can include modifications to the nucleic acid of an intron derived from a target gene.

“Exon” refers to a section of DNA which carries the coding sequence for a protein or part of it. Thus, Exons define the mRNA, which comprises 5′-non coding sequence (or UTR), protein coding sequence, and 3′-non coding sequence (or UTR). Exons are separated by intervening, non-coding sequences (introns). For purposes of the present invention, the definition of the term “exon” can include portions of exons and modifications to the nucleic acid of an exon derived from a target gene.

In some embodiments, an expression cassette of the invention can comprise a non-translated leader sequence. A number of non-translated leader sequences derived from viruses are known to enhance gene expression. Specifically, leader sequences from Tobacco Mosaic Virus (TMV, the “Ω-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968.

An expression cassette also can optionally include a transcriptional and/or translational termination region (i.e., termination region). In particular embodiments, the termination region is a transcription termination region that is functional in plants. A variety of transcriptional terminators are available for use in expression cassettes and are responsible for the termination of transcription beyond the heterologous nucleic acid of interest and correct mRNA polyadenylation. The termination region may be analogous to the transcriptional initiation region, may be analogous to the operably linked nucleic acid of interest, may be analogous to the plant host, or may be derived from another source (i.e., foreign or heterologous to the promoter or transcription regulating polynucleotide, the nucleic acid of interest, the plant host, or any combination thereof). Common transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's analogous transcription terminator can be used. In some embodiments, the terminator sequence can be the nucleic acid of SEQ ID NO:30.

An expression cassette of the invention also can include a nucleic acid encoding a screenable marker, which can be used to screen a transformed organism or cell of an organism for the presence of said marker. Many examples of screenable markers are known in the art and can be used in the expression cassettes described herein and include, but are not limited to, a nucleic acid encoding β-glucuronidase or uidA (GUS) that encodes an enzyme for which various chromogenic substrates are known; an R-locus nucleic acid that encodes a product that regulates the production of anthocyanin pigments (red color) in plant tissues (Dellaporta et al., “Molecular cloning of the maize R-nj allele by transposon-tagging with Ac,” pp. 263-282 In: Chromosome Structure and Function: Impact of New Concepts, 18th Stadler Genetics Symposium (Gustafson & Appels eds., Plenum Press 1988)); a nucleic acid encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleic acid encoding xylE that encodes a catechol dioxygenase (Zukowsky et al. (1983) Proc. Natl. Acad. Sci. USA 80:1101-1105); a nucleic acid encoding tyrosinase, an enzyme capable of oxidizing tyrosine to DOPA and dopaquinone, which in turn condenses to form melanin (Katz et al. (1983) J. Gen. Microbiol. 129:2703-2714); a nucleic acid encoding 3-galactosidase, an enzyme for which there are chromogenic substrates; a nucleic acid encoding luciferase (lux) that allows for bioluminescence detection (Ow et al. (1986) Science 234:856-859); a nucleic acid encoding aequorin, which may be employed in calcium-sensitive bioluminescence detection (Prasher et al. (1985) Biochem. Biophys. Res. Comm. 126:1259-1268); or a nucleic acid encoding green fluorescent protein (Niedz et al. (1995) Plant Cell Reports 14:403-406). One of skill in the art is capable of choosing a suitable screenable marker for use in an expression cassette of the invention.

In some embodiments, the recombinant polynucleotides described herein can be used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Vectors for use in transformation of plants and other organisms are well known in the art. Non-limiting examples of general classes of vectors include but not limited to a viral vector, a plasmid vector, a phage vector, a phagemid vector, a cosmid vector, a fosmid vector, a bacteriophage, an artificial chromosome, or an Agrobacterium binary vector in double or single stranded linear or circular form which may or may not be self transmissible or mobilizable. A vector as defined herein can transform prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some representative embodiments, the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

A non-limiting example of a vector is the plasmid pBI101 derived from the Agrobacterium tumefaciens binary vector pBIN19 allows incorporating and testing of promoters using beta-glucuronidase (GUS) expression signal (Jefferson et al, 1987, EMBO J. 6: 3901-3907). The size of the vector is 12.2 kb. It has a low-copy RK2 origin of replication and confers kanamycin resistance in both bacteria and plants. There are numerous other expression vectors known to the person skilled in the art that can be used according to the invention. Further non-limiting examples of vectors include pBIN19 (Bevan, Nucl. Acids Res. (1984)), the binary vectors pCIB200 and pCIB2001 for use with Agrobacterium, the construction of which is disclosed, for example, in WO 95133818 (example 35) (see also EP 0 332 104, example 19), the binary vector pCIB10, which contains a gene encoding kanamycin resistance for selection, the wide host-range plasmid pRK252, the construction of which is described by Rothstein et al. (Gene 53: 153-161 (1987)). Various derivatives of pCIB10 have been constructed which incorporate the gene for hygromycin B phosphotransferase are described by Gritzr et al. (Gene 25:179-188 (1983)). These derivatives enable selection of transgenic plant cells on hygromycin only (pCIB743), or hygromycin and kanamycin (pCIB715, pCIB717).

Thus, numerous transformation vectors are available for plant transformation, and the recombinant polynucleotides and expression cassettes of this invention can be used in conjunction with any such vectors. The selection of vector will depend upon the preferred transformation technique and the target species for transformation. Accordingly, in further embodiments, a recombinant polynucleotide of the invention can be comprised within a recombinant vector. The size of a vector can vary considerably depending on whether the vector comprises one or multiple expression cassettes (e.g., for molecular stacking). Thus, a vector size can range from about 3 kb to about 125 kb. Thus, in some embodiments, a vector is about 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb, 36 kb, 37 kb, 38 kb, 39 kb, 40 kb, 41 kb, 42 kb, 43 kb, 44 kb, 45 kb, 46 kb, 47 kb, 48 kb, 49 kb, 50 kb, 55 kb, 60 kb, 65 kb, 70 kb, 75 kb, 80 kb, 85 kb, 90 kb, 95 kb, 100 kb, 105 kb, 110 kb, 115 kb, 120 kb, 125 kb or any range therein, in size. In some embodiments, a vector can be about 3 kb to about 122 kb in size, about 3 kb to about 50 kb, or about 3 kb to about 10 kb.

Thus, in additional embodiments of the invention, a method of producing a transgenic cell (e.g., plant or bacterial cell) is provided, said method comprising introducing into a cell a recombinant polynucleotide and/or expression cassette of the invention. In further aspects, the invention provides a method of producing a transgenic plant cell, plant, and/or plant part, comprising introducing into said plant cell, plant or plant part a recombinant polynucleotide and/or an expression cassette of the invention, thereby producing a transgenic plant cell, plant or plant part comprising said recombinant polynucleotide or said expression cassette. In some embodiments, a transgenic plant cell comprising a recombinant polynucleotide and/or an expression cassette of the invention can be regenerated into a transgenic plant or plant part comprising said recombinant polynucleotide and/or said expression cassette of the invention in its genome. In other embodiments, wherein when the recombinant polynucleotide is operably linked to a polynucleotide of interest, the plant transformed with said recombinant nucleotide specifically or preferentially expresses the operably linked polynucleotide in the guard cells of the transformed plant.

In representative embodiments, a method of producing a transgenic plant or plant part is provided, said method comprising introducing into a plant cell an expression cassette of the invention, said expression cassette comprising a recombinant polynucleotide of the invention operatively linked to a polynucleotide of interest; regenerating a plant or plant part from said plant cell. In some embodiments, the polynucleotide of interest is expressed in said transgenic plant or plant part in a guard cell preferred or guard cell specific manner.

A further aspect of the invention provides transformed plant or bacterial cells and transformed plants and/or plant parts comprising the transformed plant cells, wherein the transformed plant cells and transformed plant and/or plant part comprise one or more recombinant polynucleotides of the invention (e.g., SEQ ID NO:1, SEQ ID NOs:10-28, or any combination thereof).

In some particular embodiments, the invention provides a transgenic plant cell comprising one or more recombinant polynucleotides of the invention and/or a transgenic plant or plant part regenerated from said transgenic plant cell. Accordingly, in some embodiments of the invention, a transgenic plant having guard cell specific or guard cell preferred expression of a polynucleotide of interest is provided, said transgenic plant regenerated from a transgenic plant cell comprising at least one recombinant polynucleotide of the invention operably linked to said polynucleotide of interest.

Any plant (or groupings of plants, for example, into a genus or higher order classification) can be employed in practicing this invention including an angiosperm, a gymnosperm, a monocot, a dicot, a C3, C4, CAM plant, a microalgae, and/or a macroalgae.

Thus, some non-limiting examples of plants that can be used with a transcription regulating polynucleotide of this invention can include vegetable crops, including artichokes, kohlrabi, arugula, leeks, asparagus, lettuce (e.g., head, leaf, romaine), bok choy, malanga, melons (e.g., muskmelon, watermelon, crenshaw, honeydew, cantaloupe), cole crops (e.g., Brussels sprouts, cabbage, cauliflower, broccoli, collards, kale, chinese cabbage, bok choy) cardoni, carrots, napa, okra, onions, celery, parsley, chick peas, parsnips, chicory, peppers, potatoes, cucurbits (e.g., marrow, cucumber, zucchini, squash, pumpkin), radishes, dry bulb onions, rutabaga, eggplant (also called brinjal), salsify, escarole, shallots, endive, garlic, spinach, green onions, squash, greens, beet (e.g., sugar beet, tropical sugar beet and fodder beet), sweet potatoes, swiss chard, horseradish, tomatoes, turnips, cassava, and spices; a fruit and/or vine crop such as apples, apricots, cherries, nectarines, peaches, pears, plums, prunes, cherry, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, and lychee; a field crop plant such as clover, alfalfa, evening primrose, meadow foam, corn/maize (field, sweet, popcorn), millet, hops, canola/rape, jojoba, peanuts, rice, safflower, small grains (rice, barley, oats, rye, wheat, etc.), sorghum, tobacco, kapok, a leguminous plant (beans, lentils, peas, soybeans), an oil plant (rape, mustard, poppy, olive, sunflower, safflower, coconut, castor oil plant, cocoa bean, groundnut), Arabidopsis, a fibre plant (cotton, flax, hemp, jute), lauraceae (cinnamon, camphor), or a plant such as coffee, sugar cane, tea, and natural rubber plants; and/or a bedding plant such as a flowering plant, a cactus, a succulent and/or an ornamental plant (e.g., orchids, carnations, roses), as well as trees such as forest (broad-leaved trees and evergreens, such as conifers), fruit, ornamental, and nut-bearing trees, as well as shrubs and other nursery stock. Other plants useful in the practice of the invention include perennial grasses, such as Arundo, switchgrass, prairie grasses, Indiangrass, Big bluestem grass, miscanthus, and the like. It is recognized that mixtures of plants can be used.

As used herein, the term “plant part” includes but is not limited to embryos, pollen, ovules, seeds, leaves, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. Further, as used herein, “plant cell” refers to a structural and physiological unit of the plant, which comprises a cell wall and also may refer to a protoplast. A plant cell of the invention can be in the form of an isolated single cell or can be a cultured cell or can be a part of a higher-organized unit such as, for example, a plant tissue or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like.

In some particular embodiments, the invention provides a transgenic seed produced from a transgenic plant of the invention, wherein the transgenic seed comprises a recombinant polynucleotide and/or expression cassette of the invention.

“Plant cell culture” means cultures of plant units such as, for example, protoplasts, cell culture cells, cells in plant tissues, pollen, pollen tubes, ovules, embryo sacs, zygotes and embryos at various stages of development. In some embodiments of the invention, a transgenic tissue culture or transgenic plant cell culture is provided, wherein the transgenic tissue or cell culture comprises a nucleic acid molecule/nucleotide sequence of the invention.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

“Introducing,” in the context of a polynucleotide sequence (e.g., a recombinant polynucleotide and/or expression cassette of the invention), means presenting a polynucleotide sequence to the plant, plant part, and/or plant cell in such a manner that the polynucleotide sequence gains access to the interior of a cell. Where more than one polynucleotide sequence is to be introduced these polynucleotide sequences can be assembled as part of a single polynucleotide or nucleic acid construct (e.g., expression cassette), or as separate polynucleotide or nucleic acid constructs (e.g., expression cassettes), and can be located on the same or different transformation vectors. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell. Transformation of a cell may be stable or transient. Thus, in some embodiments, a plant cell, plant part or plant can be stably transformed with a recombinant polynucleotide of the invention. In other embodiments, a plant cell, plant part or plant can be transiently transformed with a recombinant polynucleotide of the invention. Alternatively, a polynucleotide can be introduced into a plant by crossing a plant comprising the polynucleotide with a plant not comprising the polynucleotide. At least some members of the subsequent generation will contain the polynucleotide of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended the introduced polynucleotide is stably incorporated into the genome of the cell (, and thus the cell is stably transformed with the polynucleotide.

“Stable transformation” or “stably transformed” as used herein means that a polynucleotide is introduced into a cell and integrates into the genome of the cell. As such, the integrated polynucleotide is capable of being inherited by the progeny thereof, more particularly, by the progeny of multiple successive generations. “Genome” as used herein also includes the nuclear and the plastid genome, and therefore includes integration of a polynucleotide into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can also be detected by, for example, a Northern blot hybridization assay of RNA of the cell with nucleic acid sequences, which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

A recombinant polynucleotide of the invention (e.g., SEQ ID NO:1, SEQ ID NOs:10-28, or any combination thereof) can be introduced into a cell by any method known to those of skill in the art. In some embodiments of the invention, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hagen & Willmitzer (1988) Nucleic Acids Res. 16:9877; (2006) Transformation of Agrobacterium Using Electroporation, Cold Spring Harb Protoc, doi:10.1101/pdb.prot4665; and W, (2006) Transformation of Agrobacterium Using the Freeze-Thaw Method, Cold Spring Hart) Protoc; 2006: doi:10.1101/pdb.prot4666)

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and/or plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in particular embodiments of the invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

Likewise, the genetic properties engineered into the transgenic seeds and plants, plant parts, and/or plant cells of the invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the plant, plant part and/or plant cell in any number of ways that are well known in the art. The methods of the invention do not depend on a particular method for introducing one or more polynucleotide sequences into a plant, only that they gain access to the interior of at least one cell of the plant. Where more than one polynucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, in separate transformation events, or, for example, in plants, as part of a breeding protocol.

In some embodiments of the invention, a guard cell-specific or guard cell-preferential transcription regulating polynucleotide of the invention can be used to express traits which are especially of use for specific expression in guard-cells.

It is envisioned that many traits may be useful to be expressed in guard cells. The open reading frame to be linked to a transcription regulating polynucleotide of the invention may be obtained from an a disease resistance gene such as, for example, a bacterial disease resistance gene, a fungal disease resistance gene, a viral disease resistance gene, a nematode disease resistance gene, a nutrient utilization gene, a screenable marker gene, a gene affecting plant agronomic characteristics, i.e., yield, standability (lodging resistance), and the like, or an environment or stress resistance gene, i.e., stress tolerance or resistance (as exemplified by resistance or tolerance to drought, heat, chilling, freezing, excessive moisture, salt stress, or oxidative stress), increased yields, food content and makeup, physical appearance, drydown, standability, prolificacy, and the like. In some particular embodiments, genes of interest can include, but are not limited to, modified and unmodified ABA receptors. ABA receptors are known in the art and include, but are not limited to, the PYR/PYL/RCAR family of proteins (see, Park et al. Science 324:1068-1071 (2009); Kline et al. Plant Physiol 154:479-482 (2010); US Patent Application Publication No. 20100216643)

By “resistant” is meant a plant which exhibits substantially no phenotypic changes as a consequence of agent administration, infection with a pathogen, or exposure to stress. By “tolerant” is meant a plant which, although it may exhibit some phenotypic changes as a consequence of infection, does not have a substantially decreased reproductive capacity or substantially altered metabolism.

Thus, guard cell-preferential or guard cell-specific transcription regulating polynucleotides are useful for expressing a wide variety of genes including those which alter metabolic pathways, confer disease resistance, and the like.

The transcription regulating polynucleotides of the invention are useful to modify the phenotype of a plant. Various changes in the phenotype of a transgenic plant can be desirable (i.e., modifying the fatty acid composition of a plant, altering the amino acid content of a plant, altering a plant's pathogen defense mechanisms, and the like). These results may be achieved by providing expression of heterologous products or increased expression of endogenous products in plants. Alternatively, the results can be achieved by providing for a reduction of expression of one or more endogenous products, particularly enzymes or cofactors in the plant. These changes may also result in beneficial plant phenotypes. Generally, the transcription regulating polynucleotides described herein may be employed to express a nucleic acid segment that is operably linked to said transcription regulating polynucleotide sequences such as, for example, an open reading frame or a portion thereof, an anti-sense sequence, a sequence encoding a double-stranded RNA sequence, or a transgene.

Accordingly, in some embodiments, the invention provides a method of expressing a polynucleotide of interest in a guard cell of a plant, comprising introducing into a plant cell a recombinant polynucleotide of the invention operably linked to the polynucleotide of interest and/or an expression cassette of the invention comprising a recombinant polynucleotide of the invention operably linked to the polynucleotide of interest, regenerating the plant cell into a plant stably transformed with said recombinant polynucleotide or expression cassette of the invention, wherein the polynucleotide of interest is expressed in the guard cells of said stably transformed plant. In some embodiments of the invention, the expression of the polynucleotide of interest is guard cell specific or guard cell preferential.

In some embodiments, the transcription regulating polynucleotides of the invention can be used to confer guard cell specific or guard cell preferential expression of antisense constructs, RNAi, and the like.

In other embodiments, a method of modulating guard cell function (e.g., stomata opening and closing), comprising introducing into a plant cell a recombinant polynucleotide of the invention operably linked to a polynucleotide of interest the expression of which modulates guard cell function and/or an expression cassette of the invention comprising a recombinant polynucleotide of the invention operably linked to said polynucleotide of interest, regenerating the plant cell into a plant stably transformed with said recombinant polynucleotide of the invention, wherein the polynucleotide of interest is expressed in the guard cells of said stably transformed plant, thereby modulating the function of the guard cells of the stably transformed plant as compared to a plant that does not comprise (i.e., is not transformed with) a recombinant polynucleotide or expression cassette of the invention.

In some embodiments of the invention, the expression of the polynucleotide of interest is guard cell specific or guard cell preferential. In representative embodiments, the polynucleotide sequence of interest is a modified or unmodified ABA receptor.

In further embodiments, a method of improving plant response to water deficit and plant water use efficiency is provided, comprising introducing into a plant cell a recombinant polynucleotide of the invention operably linked to a polynucleotide of interest the expression of which can improve plant response to water deficit and plant water use efficiency and/or an expression cassette of the invention comprising a recombinant polynucleotide of the invention operably linked to said polynucleotide of interest, regenerating the plant cell into a plant stably transformed with said recombinant polynucleotide of the invention, wherein the polynucleotide of interest is expressed in the guard cells of said stably transformed plant, thereby improving response to water deficit and water use efficiency in the stably transformed plant as compared to a plant that does not comprise (i.e., is not transformed with) a recombinant polynucleotide or expression cassette of the invention. In representative embodiments, the polynucleotide sequence of interest is a modified or unmodified ABA receptor.

In still further embodiments, a method of modulating photoassimilation rate is provided, comprising introducing into a plant cell a recombinant polynucleotide of the invention operably linked to a polynucleotide of interest the expression of which can modulate photoassimilation rate and/or an expression cassette of the invention comprising a recombinant polynucleotide of the invention operably linked to said polynucleotide of interest, regenerating the plant cell into a plant stably transformed with said recombinant polynucleotide of the invention, wherein the polynucleotide of interest is expressed in the guard cells of said stably transformed plant, thereby modulating the photoassimilation rate in the stably transformed plant as compared to a plant that does not comprise (i.e., is not transformed with) a recombinant polynucleotide or expression cassette of the invention. In representative embodiments, the polynucleotide sequence of interest is a modified or unmodified ABA receptor.

In additional embodiments, a method of modulating the rate of plant transpiration, comprising introducing into a plant cell a recombinant polynucleotide of the invention operably linked to a polynucleotide of interest the expression of which can modulate the rate of plant transpiration rate and/or an expression cassette of the invention comprising a recombinant polynucleotide of the invention operably linked to said polynucleotide of interest, regenerating the plant cell into a plant stably transformed with said recombinant polynucleotide of the invention, wherein the polynucleotide of interest is expressed in the guard cells of said stably transformed plant, thereby modulating the rate of transpiration in the stably transformed plant as compared to a plant that does not comprise (i.e., is not transformed with) a recombinant polynucleotide or expression cassette of the invention. In representative embodiments, the polynucleotide sequence of interest is a modified or unmodified ABA receptor.

In a further embodiment of the invention a method of producing a plant having modulated guard cell function is provided, comprising introducing into a plant cell a recombinant polynucleotide of the invention operably linked to a polynucleotide of interest the expression of which modulates guard cell function and/or an expression cassette of the invention comprising a recombinant polynucleotide of the invention operably linked to said polynucleotide of interest, regenerating the plant cell into a plant stably transformed with said recombinant polynucleotide of the invention, thereby producing a stably transformed plant having modulated guard cell function as compared to a plant that does not comprise (i.e., is not transformed with) a recombinant polynucleotide or expression cassette of the invention.

In still further embodiments, the present invention provides plants and plant parts therefrom produced by the methods of the invention are provided wherein the transformed plant and/or plant part comprise one or more recombinant polynucleotides of the invention (e.g., e.g., SEQ ID NO:1, SEQ ID NOs:10-28) and/or an expression cassette of the invention comprising said one or more recombinant polynucleotides of the invention (e.g., e.g., SEQ ID NO:1, SEQ ID NOs:10-28). In other embodiments, the present invention further provides a seed produced from a plant of the invention, the seed comprising in its genome a recombinant polynucleotide and/or expression cassette of the invention.

This invention also is directed to methods for producing a new plant by crossing a first parent plant comprising a polynucleotide of the invention with a second parent plant. Additionally, the present invention may be used in the variety development process to derive progeny in a breeding population or crossing. Further, both first and second parent plants can be or be derived from a plant comprising a polynucleotide of the invention. A variety of breeding methods can be selected depending on the mode of reproduction, the trait, the condition of the germplasm. Thus, any such methods using a plant comprising a polynucleotide of the invention are part of this invention: selfing, backcrosses, recurrent selection, mass selection and the like.

The invention further provides a crop comprising a plurality of a plant or plants of the invention, or a progeny thereof, wherein said progeny is a transgenic plant, planted together in an agricultural field. In some embodiments, the present invention provides products produced from the plants or plant parts of the invention.

Additional aspects of the invention include a harvested product produced from the transgenic plants and/or parts thereof or crops of the invention, as well as a processed product produced from said harvested product. A harvested product can be a whole plant or any plant part, as described herein, wherein said harvested product comprises a recombinant nucleic acid molecule/construct of the invention. Thus, in some embodiments, non-limiting examples of a harvested product include a seed, a fruit, a flower or part thereof (e.g., an anther, a stigma, and the like), a leaf, a stem, and the like. In other embodiments, a processed product includes, but is not limited to, a flour, meal, oil, starch, cereal, and the like produced from a harvested seed of the invention, wherein said seed comprises a recombinant nucleic acid molecule/nucleotide sequence of the invention.

The term “modulate,” “modulates,” modulated” or “modulation” refers to enhancement (e.g., an increase) or inhibition (e.g., a reduction) in the specified activity (e.g., modulated protein production).

The terms “increase,” “increasing,” “increased,” “enhance,” “enhanced,” “enhancing,” and “enhancement,” (and grammatical variations thereof), as used herein, describe an elevation in, for example, response to water deficit and/or water use efficiency and/or an elevation in photoassimilation rate, and the like, in a plant, plant part or plant cell. This increase can be observed by comparing the increase in the plant, plant part or plant cell transformed with, for example, a recombinant polynucleotide or an expression cassette of the invention operably linked to a polynucleotide of interest, which when expressed increases a plant's response to water deficit, water use efficiency and/or photoassimilation rate, as compared to the appropriate control (e.g., the same plant, plant part or plant cell lacking (i.e., not transformed with) said recombinant polynucleotide or said expression cassette). Thus, as used herein, the terms “increase,” “increasing,” “increased,” “enhance,” “enhanced,” “enhancing,” and “enhancement” (and grammatical variations thereof), and similar terms indicate an elevation of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

As used herein, the terms “reduce,” “reduced,” “reducing,” “reduction,” “diminish,” “suppress,” and “decrease” (and grammatical variations thereof), describe, for example, a decrease in photoassimilation rate and or the rate of transpiration in a plant, plant cell and/or plant part as compared to a control as described herein. Thus, as used herein, the terms “reduce,” “reduces,” “reduced,” “reduction,” “diminish,” “suppress,” and “decrease” and similar terms mean a decrease of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or more, or any range therein, as compared to a control (e.g., the same plant, plant part or plant cell lacking (i.e., not transformed with) said recombinant polynucleotide or said expression cassette).

As used herein, the terms “improve,” “improved,” “improving,” and “improvement” (and grammatical variations thereof), describe a change, for example, in a plant's response to water deficit and/or water use efficiency that has been transformed with a recombinant polynucleotide and/or an expression cassette of the invention operably linked to a polynucleotide of interest, which when expressed modulates the transformed plant's response to water deficit and/or water use efficiency, as compared to the same plant that is not transformed with said recombinant polynucleotide or said expression cassette of the invention (i.e., a control). Depending on the desired outcome, an “improvement” can be an increase or decrease relative to a control.

All publications, patent applications, patents and other references cited herein are incorporated by reference in their entireties for the teachings relevant to the sentence and/or paragraph in which the reference is presented.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

Unless indicated otherwise, the recombinant DNA steps carried out for the purposes of the present invention, such as, for example, restriction endonuclease treatment, agarose gel electrophoresis, purification of DNA fragments, transfer of nucleic acids to nitrocellulose and nylon membranes, linking DNA fragments, transformation of E. coli cells, growing bacteria, multiplying phages and DNA sequence analysis, are carried out as described by Sambrook (Molecular Cloning: A Laboratory Manual (2^{nd} ed., Cold Spring Harbor Laboratory Press, Plainview, N.Y. (1989)). The sequencing of DNA molecules is carried out using ABI laser fluorescence DNA sequencer following the method of Sanger (Sanger, PNAS:74(12) 5463-5467(1977)).

### Example 1

### Materials and General Methods

### (A) Identification of Guard Cell Specific or Preferred Transcription Regulating Polynucleotide Sequences for

(1) Identification of the Arabidopsis thaliana At1G22960 mRNA

A sequence pile-up was carried out to define the associated mRNA sequence for At1G22960. This sequence was used in BLASTN queries of various databases to extend the transcript in both directions. In addition translation start and stop codons were identified to illustrate the gene's open reading frame. The full-length mRNA for the guard cell gene (At1G22960) was defined by assembly of several homologous cDNAs.

(2) Construction of Guard Cell Transcription Regulating Polynucleotides

Genomic sequence data from the alignments above were used to construct novel guard cell expression cassettes. SEQ ID NO:1 represents a minimal transcription regulating nucleic acid for plant guard cell transcription. SEQ ID NOs:10-16 represent a minimal transcription regulating nucleic acid plus an intron. SEQ ID NOs:17, 18, 19, 20, 21, 22, 23 represent a minimal transcription regulating nucleic acid further comprising an intron and partial sequence for an exon from the Arabidopsis At1G22960. SEQ ID NO:24 represents a minimal transcription regulating nucleic acid comprising an intron, a partial sequence for an exon from the Arabidopsis At1G22960, a tobacco mosaic virus-omega translational enhancer and a Kozak sequence. SEQ ID NO:25 and SEQ ID NO:27 represent a minimal transcription regulating nucleic acid comprising an intron, a partial sequence for an exon from the Arabidopsis At1G22960, and a Kozak sequence. SEQ ID NO:26 represents a minimal transcription regulating nucleic acid further comprising a Kozak sequence.

Nucleic acid substitutions were carried out based on cDNA/gDNA alignments as described above to remove any translation start codons and to insert translational stop codons upstream of the engineered translation start codon at key positions. The specific nucleic acid substitutions included deoxycytosine (C) substituted a, for example, position 765 and position 770 of SEQ ID NO:1, with deoxyadenine (A). Alternatively, dCTP and dATP could be used here.

### Example 2

### Vector Construction

The expression cassettes further comprise a polynucleotide of interest encoding the reporter gene β-glucuronidase (GUS) and a transcription terminator sequence (SEQ ID NO:30).

Expression cassettes representing the present invention can consist of a transcription regulating polynucleotide linked to a polynucleotide of interest and a terminator. Expression cassettes are prepared by flanking these components with appropriate restriction endonuclease sites to facilitate construction using standard recombinant DNA methodology. For example a promoter can be flanked by the restriction endonuclease sites XhoI and SanDI on the 5′-terminus and NcoI on the 3′-terminus, the gene of interest can be flanked by NcoI on the 5′-terminus and SacI on the 3′-terminus, and the terminator can be flanked by Sad on the 5′-terminus and RsrII/XmaI on the 3′-terminus. The promoter and terminator can be synthesized as single DNA product and inserted into an appropriate bacterial vector (e.g. pBlueScript™) for propagation in E. coli. The polynucleotide of interest can be inserted in between the promoter and terminus as an NcoI/SacI fragment. The complete expression cassette can be mobilized to an appropriate Agrobacterium binary vector with an appropriate SanDI or RsrII site as a SanDI/RsrII fragment.

### Example 3

### Generation of Transgenic Maize Plants

Agrobacterium binary vectors comprising a guard cell expression cassette (an expression cassette comprising a guard cell specific or preferred transcription regulating nucleic acid of the invention (e.g., SEQ ID NO:1 and/or SEQ ID NOs:10-28) fused to the plant reporter gene 3-glucuronidase (GUS) was transformed into maize.

Transformation of immature maize embryos is performed essentially as described in Negrotto et al., Plant Cell Reports 19:798-803 (2000). Various media constituents described therein can be substituted.

Agrobacterium strain LBA4404 (Invitrogen) containing the plant transformation plasmid is grown on YEP (yeast extract (5 g/L), peptone (10 g/L), NaCl (5 g/L), 15 g/l agar, pH 6.8) solid medium for 2 to 4 days at 28° C. Approximately 0.8×109 Agrobacteria are suspended in LS-inf media supplemented with 100 μM acetosyringone (As) (LSAs medium) (Negrotto et al., Plant Cell Rep 19:798-803 (2000)). Bacteria are pre-induced in this medium for 30-60 minutes.

Immature embryos from maize line, A188, or other suitable maize genotypes are excised from 8-12 day old ears into liquid LS-inf+100 μM As (LSAs). Embryos are vortexed for 5 seconds and rinsed once with fresh infection medium. Infection media is removed and Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) (Negrotto et al., Plant Cell Rep 19:798-803 (2000)) and cultured in the dark for 28° C. for 10 days.

Immature embryos producing embryogenic callus are transferred to LSD1M0.5S medium (LSDc with 0.5 mg/l 2,4-D instead of Dicamba, 10 g/l mannose, 5 g/l sucrose and no silver nitrate). The cultures are selected on this medium for 6 weeks with a subculture step at 3 weeks. Surviving calli are transferred either to LSD1M0.5S medium to be bulked-up or to Reg1 medium (as described in Negrotto et al., Plant Cell Rep 19:798-803 (2000)). Following culturing in the light (16 hour light/8 hour dark regiment), green tissues are then transferred to Reg2 medium without growth regulators (as described in Negrotto et al., Plant Cell Rep 19:798-803 (2000)) and incubated for 1-2 weeks. Plantlets are transferred to Magenta GA-7 boxes (Magenta Corp, Chicago Ill.) containing Reg3 medium (as described in Negrotto et al. (2000)) and grown in the light. Plants that were PCR positive for PMI and negative for Spectinomycin were transferred to soil and grown in the greenhouse. Plant samples of selected events are collected for GUS histochemical analysis.

### Example 4

### GUS Analysis

Quantitative GUS analysis (or enzyme activity analysis) is carried out to demonstrate and analyze the transcription regulating properties of the expression cassette using MUG (methylumbelliferyl glucuronide) as a substrate which is converted into MU (methylumbelliferone) and glucuronic acid. Under alkaline conditions this conversion can be quantitatively monitored fluorometrically (excitation at 365 nm, measurement at 455 nm; SpectroFluorimeter Thermo Life Sciences Fluoroscan) as described in (Bustos 1989) or the like.

Specifically, methods for histochemical localization of GUS enzymatic activity are as follows: (1) sample most recent fully expanded leaf at day 21; tassel leaf at R1 stage; (2) infiltrate histochemical reagent under vacuum for 30 min and repeat until tissues sink; (3) incubate the tissue in histochemical reagent for 48 hrs at 37° C. in darkness; (4) de-stain in 70% ethanol for at least 48 hrs until background is clear; and (5) photograph the tissue.

### Example 5

### Vector Construction for Overexpression and Gene “Knockout” Experiments

Vectors used to express of full-length “candidate genes” of interest in plants are designed to produce the protein of interest and are of two general types, biolistic and/or binary, depending on the plant transformation method to be used.

For biolistic transformation (biolistic vectors), the requirements are as follows:

(A) a backbone with a bacterial selectable marker (typically, an antibiotic resistance gene) and origin of replication functional in Escherichia coli (E. coli; e.g., ColE1), and

(B) a plant-specific portion consisting of:
(1) an expression cassette consisting of a transcription regulating nucleic acid of the invention (e.g., SEQ ID NO:1 and/or SEQ ID NOs:10-28), the polynucleotide of interest and a transcriptional terminator (e.g., Agrobacterium tumefaciens nos terminator or preferably the terminator encoded by the nucleic acid of SEQ ID NO:30);
(2) a plant selectable marker cassette, consisting of a suitable promoter, selectable marker gene (e.g., D-amino acid oxidase; daol) and transcriptional terminator (e.g. nos terminator).

Vectors designed for transformation by Agrobacterium tumefaciens (A. tumefaciens; binary vectors) consist of:

(A) a backbone with a bacterial selectable marker functional in both E. coli and A. tumefaciens (e.g., spectinomycin resistance mediated by the aadA gene) and two origins of replication, functional in each of aforementioned bacterial hosts, plus the A. tumefaciens virG gene;

(B) a plant-specific portion as described for biolistic vectors above, except in this instance this portion is flanked by A. tumefaciens right and left T-DNA border sequences which mediate transfer of the DNA flanked by these two sequences to the plant genome.

### Gene Silencing Vectors

Vectors designed for reducing or abolishing expression of a single gene or of a family of related genes (gene silencing vectors) are also of two general types corresponding to the methodology used to downregulate gene expression: antisense or double-stranded RNA interference (RNAi).

(A) Anti-Sense

For antisense vectors, a full-length or partial gene fragment (typically, a portion of the cDNA) can be used in the same vectors described for full-length expression, as part of the gene expression cassette. For antisense-mediated down-regulation of gene expression, the coding region of the gene or gene fragment will be in the opposite orientation relative to the transcription regulating polynucleotide of the invention; thus, mRNA will be made from the non-coding (antisense) strand in planta.

(B) RNAi

For RNAi vectors, a partial gene fragment (typically, 300 to 500 base pairs long) is used in the gene expression cassette, and is expressed in both the sense and antisense orientations, separated by a spacer region (e.g., a plant intron such as the OsSH1 intron 1, or a selectable marker, e.g., conferring kanamycin resistance). Vectors of this type are designed to form a double-stranded mRNA stem, resulting from the base pairing of the two complementary gene fragments in planta.

### Example 6

### Experimental Design

Experimental design is provided in Table 1.

TABLE 1
Experiment Design and Procedure for F1 Characterization

| Plant | | | | |
|---|---|---|---|---|
| Develop- | | | | |
| mental | Tissues | | | |
| Stage | Assayed | Assay 1 | Assay 2 | Assay 3 |
| V2-V3 | Leaf | Zygosity | GUS qRTPCR | GUS |
| | | | | ELISA |
| V4 | Leaf | GUS histochemical | | |
| | | localization | | |
| R1 | Leaf | GUS | GUS histochemical | |
| | | ELISA | localization | |
| R1 | Husk | GUS histochemical | | |
| | | localization | | |
| R1 | Cob | GUS histochemical | | |
| | | localization | | |
| R1 | Kernel | GUS histochemical | | |
| | | localization | | |
| R1 | Root | GUS histochemical | | |
| | | localization | | |
| R1 | Stem | GUS histochemical | | |
| | | localization | | |
| R3 | Kernel | GUS histochemical | | |
| | | localization | | |
| R5 | Kernel | GUS histochemical | | |
| | | localization | | |

Three representative F1 events per construct were selected for testing.

### Constructs:

(1) Construct 18620 (SEQ ID NO:24) 5′-non-transcribed sequence, 1st exon, 1st intron, part of 2nd exon; TMV-omega translational enhancer.
(2) Construct 19678 (SEQ ID NO:25) 5′-non-transcribed sequence, 1st exon, 1st intron, part of 2nd exon (no eTMV omega transcriptional enhancer).
(3) Construct 19710 (SEQ ID NO:26) 5′-non-transcribed sequence, 1st exon, (no eTMV-omega transcriptional enhancer).
(4) Construct 19738 (SEQ ID NO:27) 5′-non-transcribed sequence, 1st exon, replaced iAt1G22690 with iUbi1-13 intron, part of second exon, (no eTMV-omega transcriptional enhancer).

### (5) Construct 19711 (SEQ ID NO:28) COMPARATIVE EXAMPLE 1 (SEQ ID NO:11 of International Patent Publication WO 2008/134571 A1)

### Example 7

### Results for Vector Construct 18620 (SEQ ID NO:24)

Transgenic plants made using construct 18620 (SEQ ID NO:24) were assayed for GUS expression. The T0 plants were sampled at V8 or VT. Both ELISA and qRT-PCR were used to assess expression cassette performance. The data are summarized in Table 2.

Back-crossed seed for several events were germinated and presence of the trait gene was determined by zygosity TaqMan. Several trait positive siblings were kept and assayed for GUS activity at various stages of development (see Table 1). Data shown in Tables 2 and 4 indicate the presence of some GUS transcript and protein in leaf samples. The GUS transcript abundance as measured by qRT-PCR is very low, compared to PMI transcript abundance. This suggests the guard cell expression cassette is active in far fewer cells than the maize ubiquitin 1 cassette which was used to produce the plant selectable marker protein.

The histochemical localization data indicate the GUS protein accumulates in guard cells. The histochemical precipitate was difficult to detect (not shown) suggesting very low protein expression. We examined other plant parts for GUS accumulation and found the protein also occurs in developing kernels. The data suggested modest GUS enzyme activity was present so we quantified the protein accumulation in kernels and tassel leaf. The data are summarized in Table 3 and Table 5.

TABLE 2
Summary of GUS expression in T0 leaf samples at V8 and VT stage in select 18620 plants.

| | | | | qRT-PCR | | | |
|---|---|---|---|---|---|---|---|
| | | ELISA (ng GUS/mg | | cGUS | | cPMI | |
| | Sampling | Total protein | | Relative | | Relative | |
| Event ID | Stage | Mean | St Dev | Mean | St Dev | Mean | St Dev |
| MZDT095110A001A | V8 | 21.89 | 1.85 | 0.82 | 0.13 | 5649.19 | 394.32 |
| MZDT095110A006A | V8 | 20.89 | 3.03 | 0.69 | 0.09 | 1819.09 | 502.87 |
| MZDT095110A009A | V8 | 0.00 | 0.00 | 0.00 | 0.00 | 2893.17 | 227.62 |
| MZDT095110A024A | V8 | 40.39 | 4.01 | 2.07 | 0.37 | 5159.27 | 989.45 |
| MZDT095110B001A | V8 | 0.00 | 0.00 | 0.31 | 0.04 | 2409.64 | 226.43 |
| MZDT095110B010A | V8 | 14.92 | 0.24 | 0.62 | 0.12 | 2662.07 | 848.43 |
| MZDT095110B011A | V8 | 10.52 | 0.08 | 2.59 | 0.09 | 16085.23 | 3298.74 |
| MZDT095110B015A | V8 | 12.39 | 0.36 | 0.38 | 0.02 | 2482.36 | 1303.53 |
| MZDT095110B016A | V8 | 16.17 | 0.15 | 1.46 | 0.12 | 3166.85 | 64.24 |
| MZDT095110D006A | V8 | 13.76 | 2.04 | 1.23 | 0.13 | 3397.43 | 869.02 |
| MZDT095110D007A | V8 | 11.87 | 3.34 | 0.29 | 0.08 | 2618.39 | 211.42 |
| MZDT095110A007A | VT | 10.09 | 0.00 | 0.00 | 0.00 | 0.00 | 0.00 |
| MZDT095110A012A | VT | 18.08 | 0.17 | 0.56 | 0.06 | 2081.32 | 240.59 |
| MZDT095110A020A | VT | 15.45 | 1.30 | 0.64 | 0.06 | 4589.48 | 571.14 |
| MZDT095110A022A | VT | 16.81 | 0.09 | 0.88 | 0.08 | 4600.77 | 239.66 |
| MZDT095110B009A | VT | 17.48 | 0.15 | 2.43 | 0.18 | 39581.76 | 6968.79 |
| MZDT095110D003A | VT | 17.39 | 1.72 | 1.39 | 0.30 | 3048.68 | 213.77 |

TABLE 3
GUS protein in T1 kernels sampled at R3 from 18620 plants.

| | |
|---|---|
| | ELISA (ng GUS/ |
| Sample | mg Total Protein) |

| Event ID | Stage | Mean | St Dev |
|---|---|---|---|
| MZDT095110A020A | R3 | 0.00 | 0.00 |
| MZDT095110A001A | R3 | 105.74 | 4.97 |
| MZDT095110A006A | R3 | 0.00 | 0.00 |
| MZDT095110B011A | R3 | 0.00 | 0.00 |
| MZDT095110B016A | R3 | 32.29 | 5.30 |
| MZDT095110D007A | R3 | 0.00 | 0.00 |
| MZDT095110B015A | R3 | 70.28 | 35.13 |
| MZDT095110B009A | R4 | 89.82 | 39.38 |
| MZDT095110A012A | R4 | 74.39 | 4.04 |
| MZDT095110B012A | R4 | 0.00 | 0.00 |
| MZDT095110A001A | R4 | 62.20 | 0.00 |
| MZDT095110B011A | R4 | 35.31 | 6.23 |
| MZDT095110B015A | R4 | 44.47 | 11.43 |
| MZDT095110A024A | R4 | 58.66 | 6.15 |
| MZDT095110A006A | R4 | 56.66 | 7.81 |
| MZDT095110B016A | R4 | 21.37 | 0.00 |
| MZDT095110A020A | R4 | 39.59 | 1.61 |
| MZDT095110B012A | R5 | 0.00 | 0.00 |

TABLE 4
Leaf GUS qRT and ELISA summary at V3 stage.

| | | | | | | | ELISA (ng | |
|---|---|---|---|---|---|---|---|---|
| | | | Gus qRT-PCR | | PMI qRT-PCR | | GUS/mg Total | |
| | Plant | Sampling | Relative | | Relative | | Protein) | |
| Event ID | Number | Stage | Mean | St Dev | Mean | St Dev | Mean | St Dev |
| MZDT095110B009A | 1 | V3 | 2.55 | 0.53 | 51765.43 | 3709.21 | 2.84 | 0.23 |
| MZDT095110B009A | 2 | V3 | 21.98 | 2.26 | 95265.57 | 9037.00 | 2.55 | 0.07 |
| MZDT095110B009A | 3 | V3 | 18.23 | 5.65 | 85698.85 | 13939.57 | 2.87 | 0.11 |
| MZDT095110B009A | 4 | V3 | 26.72 | 2.14 | 102286.65 | 8829.55 | 2.74 | 0.29 |
| MZDT095110B009A | 5 | V3 | 0.00 | 0.00 | 0.00 | 0.00 | ND | ND |
| MZDT095110B009A | 6 | V3 | 31.50 | 7.08 | 198860.79 | 19415.63 | 2.68 | 0.29 |
| MZDT095110B009A | 7 | V3 | 7.88 | 2.69 | 67297.45 | 13791.10 | 3.44 | 1.00 |
| MZDT095110A006A | 1 | V3 | 27.57 | 3.25 | 12569.20 | 939.55 | 4.29 | 0.15 |
| MZDT095110A006A | 2 | V3 | 0.00 | 0.00 | 0.00 | 0.00 | ND | ND |
| MZDT095110A006A | 3 | V3 | 10.55 | 1.85 | 16153.69 | 2157.03 | 3.40 | 0.72 |
| MZDT095110A006A | 4 | V3 | 3.80 | 2.36 | 12333.04 | 1305.20 | 2.64 | 0.46 |
| MZDT095110A006A | 5 | V3 | 41.45 | 2.20 | 11813.73 | 1224.10 | 2.63 | 0.85 |
| MZDT095110A006A | 6 | V3 | 0.00 | 0.00 | 10223.93 | 1296.74 | 2.01 | 0.91 |
| MZDT095110A006A | 7 | V3 | 26.39 | 2.50 | 11336.77 | 1859.82 | 3.05 | 0.63 |
| MZDT095110A001A | 1 | V3 | 4.43 | 0.70 | 24379.84 | 664.91 | 3.13 | 1.51 |
| MZDT095110A001A | 2 | V3 | 30.17 | 1.47 | 69325.35 | 5095.89 | 2.64 | 0.86 |
| MZDT095110A001A | 3 | V3 | 0.00 | 0.00 | 0.00 | 0.00 | 1.25 | ND |
| MZDT095110A001A | 4 | V3 | 35.93 | 6.49 | 37165.34 | 2762.14 | 3.02 | 0.92 |
| MZDT095110A001A | 5 | V3 | 32.14 | 1.75 | 52787.56 | 4830.77 | 2.39 | 0.96 |
| MZDT095110A001A | 6 | V3 | 30.02 | 2.16 | 60794.25 | 1301.49 | 2.11 | 0.36 |
| MZDT095110A001A | 7 | V3 | 32.14 | 5.62 | 46057.78 | 3005.25 | 2.05 | 0.22 |

TABLE 5
Summary of GUS expression in T1 tassel leaf
sampled at R1 in select 18620 plants.

| | | |
|---|---|---|
| | Sam- | ELISA (ng GUS/ |
| Plant | pling | mg Total Protein) |

| Event ID | Number | Stage | Mean | St Dev |
|---|---|---|---|---|
| MZDT095110B009A | 1 | R1 | 2.84 | NA |
| MZDT095110B009A | 2 | R1 | 2.55 | NA |
| MZDT095110B009A | 3 | R1 | 2.87 | NA |
| MZDT095110B009A | 4 | R1 | 2.74 | NA |
| MZDT095110B009A | 5 | R1 | ND | NA |
| MZDT095110B009A | 6 | R1 | 2.68 | NA |
| MZDT095110B009A | 7 | R1 | 3.44 | NA |
| MZDT095110A006A | 1 | R1 | 4.29 | NA |
| MZDT095110A006A | 2 | R1 | ND | NA |
| MZDT095110A006A | 3 | R1 | 3.40 | NA |
| MZDT095110A006A | 4 | R1 | 2.64 | NA |
| MZDT095110A006A | 5 | R1 | 2.63 | NA |
| MZDT095110A006A | 6 | R1 | 2.01 | NA |
| MZDT095110A006A | 7 | R1 | 3.05 | NA |
| MZDT095110A001A | 1 | R1 | 3.13 | NA |
| MZDT095110A001A | 2 | R1 | 2.64 | NA |
| MZDT095110A001A | 3 | R1 | 1.25 | NA |
| MZDT095110A001A | 4 | R1 | 3.02 | NA |
| MZDT095110A001A | 5 | R1 | 2.39 | NA |
| MZDT095110A001A | 6 | R1 | 2.11 | NA |
| MZDT095110A001A | 7 | R1 | 2.05 | NA |

In summary, the 18620 expression cassette (SEQ ID NO:13) drives GUS protein accumulation specifically in leaf guard cells, however, histochemical localization data indicate that GUS protein was not in every guard cell. Expression in other tissues was investigated in different T1 tissues such as husk, cob, stem, root, tassel and kernel. The GUS protein was detected in cob and kernels.

### Example 8

### Results for Vector Construct 19678 (SEQ ID NO:25)

Transgenic plants made using construct 19678 (SEQ ID NO:25) were assayed for GUS expression. The T0 plants were sampled at V3 or R1. Both ELISA and qRT-PCR were used to assess expression cassette performance. The data are summarized in Tables 6 and 7.

Back-crossed seed for several events were germinated and presence of the trait gene was determined by zygosity TaqMan. Several trait positive siblings were kept and assayed for GUS activity at various stages of development (see Table 1). Data shown in Tables 8 and 9 indicate the presence of some GUS transcript and protein in leaf samples. The GUS transcript abundance as measured by qRT-PCR is very low, compared to PMI transcript abundance. This suggests the guard cell expression cassette is active in far fewer cells than the maize ubiquitin 1 cassette which was used to produce the plant selectable marker protein.

Our histochemical localization data indicate the GUS protein accumulates in guard cells. The histochemical precipitate was difficult to detect (not shown) suggesting very low protein expression. We examined other plant parts for GUS accumulation and found the protein also occurs in developing kernels.

TABLE 6
Summary of GUS expression in T0 leaf
sampled at V3 in select 19678 plants.

| | Sam- | GUS qRT-PCR | | ELISA (ng GUS/ |
|---|---|---|---|---|
| | pling | Relative | | mg Total Protein) |
| Event ID | Stage | Mean | St Dev | Mean |
| MZDT104612A008A | V3 | 16.79 | 1.20 | 0.00 |
| MZDT104612A017A | V3 | 30.43 | 1.10 | 0.00 |
| MZDT104612A024A | V3 | 3402.39 | 81.18 | 0.00 |
| MZDT104612A026A | V3 | 44.01 | 4.01 | 0.00 |
| MZDT104612A038A | V3 | 28.43 | 3.35 | 0.00 |
| MZDT104612A039A | V3 | 23.17 | 0.17 | 0.00 |
| MZDT104612A064A | V3 | 40.85 | 9.88 | 0.00 |
| MZDT104612A068A | V3 | 51.03 | 4.22 | 0.00 |
| MZDT104612A074A | V3 | 0.00 | 0.00 | 0.00 |
| MZDT104612A005A | V3 | 11.06 | 4.68 | 0.55 |
| MZDT104612A076A | V3 | 102.35 | 8.75 | 1.40 |
| MZDT104612A048A | V3 | 39.28 | 7.51 | 1.45 |
| MZDT104612A058A | V3 | 55.81 | 8.86 | 2.36 |
| MZDT104612A081A | V3 | 60.89 | 8.56 | 2.45 |
| MZDT104612A034A | V3 | 58.05 | 3.02 | 2.46 |
| MZDT104612A007A | V3 | 13.42 | 1.43 | 3.53 |
| MZDT104612A032A | V3 | 28.59 | 1.61 | 4.67 |
| MZDT104612A063A | V3 | 28.81 | 2.42 | 5.01 |
| MZDT104612A025A | V3 | 83.96 | 3.99 | 5.70 |
| MZDT104612A019A | V3 | 65.82 | 4.08 | 6.56 |
| MZDT104612A083A | V3 | 320.53 | 21.03 | 6.97 |
| MZDT104612A001A | V3 | 173.87 | 23.62 | 7.55 |
| MZDT104612A014A | V3 | 29.50 | 1.92 | 8.46 |
| MZDT104612A015A | V3 | 61.77 | 6.05 | 8.61 |
| MZDT104612A022A | V3 | 17.29 | 0.40 | 13.24 |
| MZDT104612A004A | V3 | 94.02 | 12.94 | 14.07 |
| MZDT104612A054A | V3 | 86.57 | 7.42 | 18.22 |
| MZDT104612A036A | V3 | 118.08 | 11.36 | 21.32 |
| MZDT104612A075A | V3 | 667.40 | 29.60 | 26.07 |
| MZDT104612A078A | V3 | 0.00 | 0.00 | 32.31 |
| MZDT104612A023A | V3 | 72.75 | 7.40 | 33.67 |

TABLE 7
Summary of GUS expression in T0 tassel leaf
sampled at R1 in select 19678 plants.

| | |
|---|---|
| Sam- | ELISA (ng GUS/ |
| pling | mg Total Protein) |

| Event ID | Stage | Mean | St Dev |
|---|---|---|---|
| MZDT104612A078A | R1 | ND | ND |
| MZDT104612A007A | R1 | 1.54 | 0.78 |
| MZDT104612A076A | R1 | 13.11 | 1.12 |
| MZDT104612A064A | R1 | 3.76 | 3.26 |
| MZDT104612A048A | R1 | 15.28 | 0.64 |
| MZDT104612A005A | R1 | 17.54 | 16.6 |
| MZDT104612A058A | R1 | 18.1 | 1.92 |
| MZDT104612A032A | R1 | 18.93 | 1.04 |
| MZDT104612A081A | R1 | 18.98 | 3.72 |
| MZDT104612A004A | R1 | 21.93 | 1.5 |
| MZDT104612A014A | R1 | 28.76 | 0.74 |
| MZDT104612A001A | R1 | 33.52 | 2.45 |

TABLE 8
Summary of GUS and PMI expression in T1 leaf sampled at V3 in select 19678 plants.

| | | | Gus (qRT-PCR) | | | | ELISA | |
|---|---|---|---|---|---|---|---|---|
| | | | Relative | | | | (ng GUS/mg | |
| | Plant | Sampling | Relative | | PMI (qRT-PCR) | | Total Protein) | |
| Event ID | Number | Stage | Mean | St Dev | Mean | St Dev | Mean | St Dev |
| MZDT104612A078A | 1 | V3 | 0.00 | 0.00 | 32652.03 | 3954.71 | ND | NA |
| MZDT104612A078A | 2 | V3 | 5.74 | 0.91 | 38542.68 | 1955.71 | ND | NA |
| MZDT104612A078A | 3 | V3 | 0.00 | 0.00 | 0.00 | 0.00 | ND | NA |
| MZDT104612A078A | 4 | V3 | 9.42 | 1.76 | 45393.76 | 3336.24 | ND | NA |
| MZDT104612A078A | 5 | V3 | 0.00 | 0.00 | 29993.67 | 3437.10 | ND | NA |
| MZDT104612A078A | 6 | V3 | 0.00 | 0.00 | 34162.48 | 2247.13 | ND | NA |
| MZDT104612A078A | 7 | V3 | 0.00 | 0.00 | 35771.46 | 1767.42 | ND | NA |
| MZDT104612A014A | 1 | V3 | 55.43 | 1.44 | 108153.23 | 5888.13 | 7.84 | 2.63 |
| MZDT104612A014A | 2 | V3 | 0.00 | 0.00 | 0.00 | 0.00 | ND | NA |
| MZDT104612A014A | 3 | V3 | 34.13 | 3.38 | 27515.39 | 5505.52 | 2.93 | 2.61 |
| MZDT104612A014A | 4 | V3 | 17.74 | 2.22 | 26339.57 | 2255.91 | 3.57 | 0.09 |
| MZDT104612A014A | 5 | V3 | 29.54 | 3.70 | 26076.53 | 1718.67 | 3.85 | 1.07 |
| MZDT104612A014A | 6 | V3 | 32.55 | 2.30 | 24104.77 | 1030.88 | 4.62 | 0.25 |
| MZDT104612A014A | 7 | V3 | 28.52 | 2.39 | 21235.72 | 2749.32 | 3.97 | 0.60 |
| MZDT104612A001A | 1 | V3 | 57.81 | 4.04 | 10310.27 | 1006.31 | 7.65 | 1.79 |
| MZDT104612A001A | 2 | V3 | 83.74 | 4.68 | 9954.11 | 454.37 | 8.61 | 1.81 |
| MZDT104612A001A | 3 | V3 | 96.84 | 6.12 | 11521.30 | 746.53 | 5.56 | 1.42 |
| MZDT104612A001A | 4 | V3 | 0.00 | 0.00 | 0.00 | 0.00 | ND | NA |
| MZDT104612A001A | 5 | V3 | 64.32 | 0.87 | 8800.54 | 1197.25 | 4.07 | 0.31 |
| MZDT104612A001A | 6 | V3 | 64.19 | 11.44 | 9888.49 | 1447.51 | 5.96 | 0.60 |
| MZDT104612A001A | 7 | V3 | 73.19 | 2.06 | 5664.87 | 837.84 | 5.70 | 0.22 |

TABLE 9
Summary of GUS expression in T1 tassel
leaf sampled at R1 in select 19678

| | | |
|---|---|---|
| | Sam- | ELISA (ng GUS/ |
| Plant | pling | mg Total Protein) |

| Event ID | Number | Stage | Mean | St Dev |
|---|---|---|---|---|
| MZDT104612A078A | 1 | R1 | 0.82 | NA |
| MZDT104612A078A | 2 | R1 | 1.16 | NA |
| MZDT104612A078A | 3 | R1 | 1.05 | NA |
| MZDT104612A078A | 4 | R1 | 0.98 | NA |
| MZDT104612A078A | 5 | R1 | 1.83 | NA |
| MZDT104612A078A | 6 | R1 | 14.28 | NA |
| MZDT104612A078A | 7 | R1 | 1.24 | NA |
| MZDT104612A014A | 1 | R1 | 2.14 | NA |
| MZDT104612A014A | 2 | R1 | 0.87 | NA |
| MZDT104612A014A | 3 | R1 | ND | NA |
| MZDT104612A014A | 4 | R1 | 1.65 | NA |
| MZDT104612A014A | 5 | R1 | 2.32 | NA |
| MZDT104612A014A | 6 | R1 | 2.76 | NA |
| MZDT104612A014A | 7 | R1 | 3.64 | NA |
| MZDT104612A001A | 1 | R1 | 5.50 | NA |
| MZDT104612A001A | 2 | R1 | 2.36 | NA |
| MZDT104612A001A | 3 | R1 | 1.74 | NA |
| MZDT104612A001A | 4 | R1 | ND | NA |
| MZDT104612A001A | 5 | R1 | 3.50 | NA |
| MZDT104612A001A | 6 | R1 | 2.55 | NA |
| MZDT104612A001A | 7 | R1 | 4.06 | NA |

In summary, the 19678 construct (SEQ ID NO:25) drives GUS protein accumulation specifically in leaf guard cells, however, histochemical localization data indicate that GUS protein was not in every guard cell. The expression pattern was similar to construct 18620 (SEQ ID NO:25). The quantitative evidence suggests that eliminating the tobacco mosaic virus omega translational enhancer increases GUS protein accumulation by 2-3-fold. Expression in other tissues was investigated in different T1 tissues such as husk, cob, stem, root, tassel and kernel. The GUS protein was detected in cob and kernels.

### Example 9

### Results for Vector Construct 19738 (SEQ ID NO:27)

Transgenic plants made using construct 19738 (SEQ ID NO:27) were assayed for GUS expression. The T0 plants were sampled at V3 or R1. Both ELISA and qRT-PCR were used to assess expression cassette performance. The data are summarized in Table 10 and Table 11.

Back-crossed seed for several events were germinated and presence of the trait gene was determined by zygosity TaqMan. Several trait positive siblings were kept and assayed for GUS activity at various stages of development (see Table 1). Data shown in Table 12 and Table 13 indicate the presence of some GUS transcript and protein in leaf samples. The GUS transcript abundance as measured by qRT-PCR is low, compared to PMI transcript abundance. This suggests the guard cell expression cassette is active in fewer cells than the maize ubiquitin 1 cassette which was used to produce the plant selectable marker protein.

Histochemical localization data indicate the GUS protein accumulates in guard cells as well as other cells. The histochemical precipitate was easy to detect (not shown) suggesting modest protein expression. We examined other plant parts for GUS accumulation and found the protein also occurs in other tissues including husk, cob, stem, root, tassel and kernel.

TABLE 10
Summary of GUS expression in T0 leaf
sampled at V3 in select 19738 plants.

| | Sam- | GUS qRT-PCR | | ELISA (ng GUS/ |
|---|---|---|---|---|
| | pling | Relative | | mg Total Protein) |
| Event ID | Stage | Mean | St Dev | Mean |
| MZDT104800B028A | V3 | 32.61 | 3.26 | 55.33 |
| MZDT104800B004A | V3 | 169.26 | 19.13 | 56.03 |
| MZDT104800B040A | V3 | 153.83 | 25.25 | 58.89 |
| MZDT104800A040A | V3 | 109.10 | 7.97 | 75.44 |
| MZDT104800A037A | V3 | 175.51 | 46.34 | 94.71 |
| MZDT104800B025A | V3 | 213.01 | 9.21 | 96.08 |
| MZDT104800B011A | V3 | 270.42 | 29.61 | 102.77 |
| MZDT104800A004A | V3 | 436.19 | 49.47 | 102.94 |
| MZDT104800B017A | V3 | 306.28 | 1.99 | 106.88 |
| MZDT104800B002A | V3 | 466.67 | 12.75 | 117.12 |
| MZDT104800A032A | V3 | 248.19 | 29.21 | 140.45 |
| MZDT104800B042A | V3 | 235.50 | 23.47 | 168.52 |
| MZDT104800B022A | V3 | 931.67 | 54.67 | 193.41 |
| MZDT104800A046A | V3 | 328.88 | 10.26 | 198.95 |
| MZDT104800A003A | V3 | 391.76 | 28.36 | 208.70 |
| MZDT104800B021A | V3 | 1083.92 | 41.34 | 214.81 |
| MZDT104800A045A | V3 | 580.52 | 134.27 | 218.59 |
| MZDT104800B007A | V3 | 511.11 | 5.04 | 263.98 |
| MZDT104800A031A | V3 | 324.21 | 7.30 | 293.34 |
| MZDT104800B006A | V3 | 815.21 | 17.31 | 460.55 |
| MZDT104800B018A | V3 | 3320.75 | 500.25 | 905.88 |
| MZDT110207A040A | V3 | 0.00 | 0.00 | 2.81 |
| MZDT110207A050A | V3 | 90.86 | 4.22 | 44.89 |
| MZDT110207A079A | V3 | 59.30 | 5.69 | 47.54 |
| MZDT110207A023A | V3 | 111.89 | 3.75 | 49.56 |
| MZDT110207A046A | V3 | 209.67 | 60.63 | 52.29 |
| MZDT110207A103A | V3 | 60.22 | 11.05 | 52.40 |
| MZDT110207A051A | V3 | 576.54 | 25.21 | 62.61 |
| MZDT110207A080A | V3 | 115.88 | 3.90 | 71.85 |
| MZDT110207A076A | V3 | 109.51 | 14.81 | 79.93 |
| MZDT110207A028A | V3 | 0.00 | 0.00 | 81.19 |
| MZDT110207A022A | V3 | 79.28 | 10.87 | 86.48 |
| MZDT110207A108A | V3 | 112.75 | 9.45 | 87.07 |
| MZDT110207A100A | V3 | 166.39 | 9.42 | 90.68 |
| MZDT110207A045A | V3 | 84.46 | 5.62 | 93.71 |
| MZDT110207A034A | V3 | 120.21 | 16.32 | 101.42 |
| MZDT110207A106A | V3 | 75.81 | 14.03 | 106.50 |
| MZDT110207A099A | V3 | 106.58 | 15.21 | 148.60 |
| MZDT110207A026A | V3 | 175.61 | 18.62 | 164.95 |
| MZDT110207A102A | V3 | 213.59 | 32.67 | 176.27 |
| MZDT110207A025A | V3 | 122.89 | 29.94 | 194.62 |
| MZDT110207A003A | V3 | 686.59 | 74.71 | 495.43 |
| MZDT110207A004A | V3 | 74.18 | 6.72 | 80.15 |

TABLE 11
Summary of GUS expression in T0 tassel leaf
sampled at R1 in select 19738 plants.

| | |
|---|---|
| Sam- | ELISA (ng GUS/ |
| pling | mg Total Protein) |

| Event ID | Stage | Mean | St Dev |
|---|---|---|---|
| MZDT104800B040A | R1 | 36.45 | 5.92 |
| MZDT110207A040A | R1 | 39.56 | 2.33 |
| MZDT104800A032A | R1 | 53.27 | 0.14 |
| MZDT104800A037A | R1 | 58.26 | 8.88 |
| MZDT104800B028A | R1 | 60.82 | 21.81 |
| MZDT104800B004A | R1 | 75.99 | 1.61 |
| MZDT110207A004A | R1 | 77.94 | 2.68 |
| MZDT110207A045A | R1 | 104.12 | 4.07 |
| MZDT104800B011A | R1 | 109.09 | 5.71 |
| MZDT104800B025A | R1 | 114.49 | 13.54 |
| MZDT104800B017A | R1 | 127.53 | 1.74 |
| MZDT104800B042A | R1 | 130.23 | 13.76 |
| MZDT104800B021A | R1 | 132.31 | 47.90 |
| MZDT104800A046A | R1 | 152.60 | 19.88 |
| MZDT104800B002A | R1 | 154.44 | 24.18 |
| MZDT110207A003A | R1 | 174.59 | 18.16 |
| MZDT104800B007A | R1 | 216.47 | 35.55 |
| MZDT104800A031A | R1 | 231.88 | 17.59 |
| MZDT104800B006A | R1 | 279.07 | 29.16 |
| MZDT104800B018A | R1 | 284.00 | 38.40 |
| MZDT104800B022A | R1 | 393.39 | 161.38 |

TABLE 12
Summary of GUS expression in T1 leaf sampled at V3 in select 19738 plants.

| | | | GUS qRT-PCR | | PMI qRT-PCR | | ELISA (ng GUS/mg | |
|---|---|---|---|---|---|---|---|---|
| | Plant | Sampling | Relative | | Relative | | Total Protein) | |
| Event ID | Number | Stage | Mean | St Dev | Mean | St Dev | Mean | St Dev |
| MZDT104800A045A | 1 | V3 | 77.83 | 9.53 | 10531.1 | 1104.34 | 31.38 | 8.26 |
| MZDT104800A045A | 2 | V3 | 136.26 | 21.8 | 14744.4 | 1706.46 | 24.66 | 23.6 |
| MZDT104800A045A | 3 | V3 | 94.33 | 3.99 | 11205.3 | 457.5 | 29.83 | 20.81 |
| MZDT104800A045A | 4 | V3 | 120.16 | 13.55 | 27630.08 | 2925.51 | 33.04 | 12.52 |
| MZDT104800A045A | 5 | V3 | 103.2 | 6.63 | 12420.78 | 868.90 | 29.27 | 7.87 |
| MZDT104800A045A | 6 | V3 | 151.79 | 7.46 | 18986.14 | 1320.79 | 27.38 | 5.87 |
| MZDT104800A045A | 7 | V3 | 0 | 0 | 0 | 0 | ND | ND |

TABLE 13
Summary of GUS expression in T1 tassel leaf
sampled at R1 in select 19738 plants

| | | |
|---|---|---|
| | Sam- | ELISA (ng GUS/ |
| Plant | pling | mg Total Protein) |

| Event ID | Number | Stage | Mean | St Dev |
|---|---|---|---|---|
| MZDT104800A045A | 1 | R1 | 31.38 | NA |
| MZDT104800A045A | 2 | R1 | 24.66 | NA |
| MZDT104800A045A | 3 | R1 | 29.83 | NA |
| MZDT104800A045A | 4 | R1 | 33.04 | NA |
| MZDT104800A045A | 5 | R1 | 29.27 | NA |
| MZDT104800A045A | 6 | R1 | 27.38 | NA |
| MZDT104800A045A | 7 | R1 | ND | NA |

In summary, the 19738 expression cassette (SEQ ID NO:27) drives GUS protein accumulation in leaf guard cells as well as other cells. The histochemical localization data indicate that GUS protein was present in almost every guard cell (not shown). The quantitative evidence suggests that substituting the maize ubiquitin 1 intron for the At1G22690 intron increases GUS protein accumulation by several-fold. It also increases expression in other tissues. This was investigated in different T1 tissues such as husk, cob, stem, root, tassel and kernel. The GUS protein was detected in all tissues examined.

### Example 10

### Results for Vector Construct 19710 (SEQ ID NO:26)

Transgenic plants made using construct 19710 (SEQ ID NO:26) were assayed for GUS expression. The T0 plants were sampled at V3 or R1. Both ELISA and qRT-PCR were used to assess expression cassette performance. The data are summarized in Table 14 and Table 15.

Back-crossed seed for several events were germinated and presence of the trait gene was determined by zygosity TaqMan. Several trait positive siblings were kept and assayed for GUS activity at various stages of development (see Table 1). Data shown in Table 16 indicates the presence of almost no GUS transcript and protein in leaf samples. The GUS transcript abundance as measured by qRT-PCR is extremely low, compared to PMI transcript abundance. This suggests the guard cell expression cassette is active in fewer cells than the maize ubiquitin 1 cassette which was used to produce the plant selectable marker protein.

Histochemical localization data indicate no detectable GUS protein accumulation in guard cells or other cells (not shown). We examined other plant parts for GUS accumulation and found no evidence the protein occurs in other tissues including husk, cob, stem, root, tassel and kernel.

TABLE 14
Summary of GUS and PMI expression in T0 leaf
sampled at V3 in select 19710 plants.

| | Sam- | GUS qRT-PCR | | PMI qRT-PCR | |
|---|---|---|---|---|---|
| | pling | Relative | | Relative | |
| Event ID | Stage | Mean | St Dev | Mean | St Dev |
| MZDT104601B033A | V3 | 0.00 | 0.00 | 9051.32 | 1196.32 |
| MZDT104601B014A | V3 | 0.00 | 0.00 | 15.58 | 2.36 |
| MZDT104601B058A | V3 | 0.00 | 0.00 | 0.00 | 0.00 |
| MZDT104601A003A | V3 | 0.00 | 0.00 | 4845.95 | 4845.95 |
| MZDT104601A006A | V3 | 0.00 | 0.00 | 0.00 | 0.00 |
| MZDT104601B001A | V3 | 0.00 | 0.00 | ND | 0.00 |
| MZDT104601B002A | V3 | 0.00 | 0.00 | 43088.23 | 5551.95 |
| MZDT104601B009A | V3 | 0.00 | 0.00 | 13869.26 | 1753.43 |
| MZDT104601B010A | V3 | 0.00 | 0.00 | 13822.15 | 753.74 |
| MZDT104601B025A | V3 | 29.99 | 7.73 | 8647.60 | 3085.31 |
| MZDT104601B027A | V3 | 0.00 | 0.00 | 4819.97 | 394.21 |
| MZDT104601B034A | V3 | 0.00 | 0.00 | 24327.77 | 4178.94 |
| MZDT104601B038A | V3 | 0.00 | 0.00 | 7.67 | 0.95 |
| MZDT104601B039A | V3 | 0.00 | 0.00 | ND | 0.00 |
| MZDT104601B043A | V3 | 0.00 | 0.00 | 9104.00 | 478.44 |
| MZDT104601B047A | V3 | 0.00 | 0.00 | 4179.03 | 448.59 |
| MZDT104601B050A | V3 | 0.00 | 0.00 | 16.30 | 3.00 |
| MZDT104601B056A | V3 | 0.00 | 0.00 | ND | 0.00 |
| MZDT104601B057A | V3 | 0.00 | 0.00 | 5637.78 | 1078.99 |
| MZDT104601B059A | V3 | 135.38 | 13.27 | 6344.63 | 813.18 |
| MZDT104601B060A | V3 | 115.58 | 10.38 | 2836.49 | 327.24 |
| MZDT104601B064A | V3 | 0.00 | 0.00 | 2746.34 | 251.67 |
| MZDT104601B068A | V3 | 0.00 | 0.00 | 7575.01 | 845.64 |
| MZDT104601B069A | V3 | 0.00 | 0.00 | ND | 0.00 |
| MZDT104601B072A | V3 | 0.00 | 0.00 | 117677.44 | 19541.36 |
| MZDT104601B080A | V3 | 0.00 | 0.00 | 16820.39 | 939.59 |
| MZDT104601B083A | V3 | 0.00 | 0.00 | 23075.13 | 2598.29 |
| MZDT104601B086A | V3 | 0.00 | 0.00 | 9642.84 | 1076.23 |

TABLE 15
Summary of GUS expression in T0 tassel leaf
sampled at R1 in select 19710 plants

| | |
|---|---|
| Sam- | ELISA (ng GUS/ |
| pling | mg total protein) |

| Event ID | Stage | Mean | St Dev |
|---|---|---|---|
| MZDT104601B014A | R1 | 0.00 | 0.00 |
| MZDT104601B033A | R1 | 0.00 | 0.00 |
| MZDT104601B058A | R1 | 3.48 | 0.72 |
| MZDT104601A006A | R1 | 3.98 | 0.81 |
| MZDT104601B059A | R1 | 4.75 | 0.58 |
| MZDT104601B027A | R1 | 5.36 | 0.36 |
| MZDT104601B009A | R1 | 6.17 | 0.90 |
| MZDT104601B043A | R1 | 6.49 | 0.32 |
| MZDT104601B060A | R1 | 6.64 | 3.09 |
| MZDT104601B010A | R1 | 15.99 | 4.77 |

TABLE 16
Summary of GUS and PMI expression in T1 leaf sampled at V3 in select 19710 plants

| | | | Gus qRT-PCR | | PMI qRT-PCR | | ELISA |
|---|---|---|---|---|---|---|---|
| | | Sampling | Relative | St | Relative | St | (ng GUS/mg |
| Event ID | Plant # | Stage | Mean | Dev | Mean | Dev | total protein) |
| MZDT104601B010A | 1 | V3 | 10.13 | 1.11 | 9068.46 | 893.08 | ND |
| MZDT104601B010A | 2 | V3 | 0.00 | 0.00 | 4108.77 | 256.71 | 4.71 |
| MZDT104601B010A | 3 | V3 | 0.00 | 0.00 | 46.88 | 3.14 | ND |
| MZDT104601B010A | 4 | V3 | 0.00 | 0.00 | 8455.22 | 1970.81 | ND |
| MZDT104601B010A | 5 | V3 | 0.00 | 0.00 | 22.27 | 1.66 | ND |
| MZDT104601B010A | 6 | V3 | 9.29 | 1.16 | 11565.65 | 640.56 | ND |
| MZDT104601B010A | 7 | V3 | 8.96 | 0.62 | 6120.29 | 521.45 | ND |
| MZDT104601B010A | 8 | V3 | 6.86 | 1.12 | 5604.12 | 393.87 | ND |

In summary, the 19710 expression cassette (SEQ ID NO:26) is essentially inactive in maize. The evidence suggests that eliminating the At1G22690 intron from the expression cassette renders the construct non-functional in maize. Expression analysis was not conducted beyond V3.

### Example 11

### Comparative Example 1—Vector Construct 19711 (SEQ ID NO:28)

Transgenic plants made using construct 19711 (SEQ ID NO:28) were assayed for GUS expression. The T0 plants were sampled at V3 or R1. Both ELISA and qRT-PCR were used to assess expression cassette performance. The data are summarized in Table 17 and Table 18.

Back-crossed seed for several events were germinated and presence of the trait gene was determined by zygosity TaqMan. Several trait positive siblings were kept and assayed for GUS activity at various stages of development (see Table 1). Data shown in Table 19 and Table 20 indicate the presence of almost no GUS transcript and protein in leaf samples. The GUS transcript abundance as measured by qRT-PCR is extremely low, compared to PMI transcript abundance. This suggests the guard cell expression cassette is active in fewer cells than the maize ubiquitin 1 cassette which was used to produce the plant selectable marker protein.

Histochemical localization data indicate no detectable GUS protein accumulation in guard cells or other cells (not shown). We examined other plant parts for GUS accumulation and found no evidence the protein occurs in other tissues including husk, cob, stem, root, tassel and kernel.

TABLE 17
Summary of GUS and PMI expression in T0
leaf sampled at V3 in select 19711 plants

| | Sam- | GUS qRT-PCR | | |
|---|---|---|---|---|
| | pling | Relative | | ELISA (ng GUS/ |
| Event ID | Stage | Mean | St Dev | mg total protein) |
| MZDT110400A005A | V3 | 0.00 | 0.00 | 0.00 |
| MZDT110400A009A | V3 | 5.22 | 2.14 | 0.00 |
| MZDT110400A012A | V3 | 0.00 | 0.00 | 0.00 |
| MZDT110400A013A | V3 | 74.74 | 10.10 | 0.00 |
| MZDT110400A015A | V3 | 0.00 | 0.00 | 0.00 |
| MZDT110400A016A | V3 | 0.00 | 0.00 | 0.00 |
| MZDT110400A018A | V3 | 120.17 | 14.63 | 0.00 |
| MZDT110400A020A | V3 | 0.00 | 0.00 | 0.00 |
| MZDT110400A021A | V3 | 38.78 | 5.79 | 0.00 |
| MZDT110400A027A | V3 | 0.00 | 0.00 | 0.00 |
| MZDT110400A028A | V3 | 0.00 | 0.00 | 0.00 |
| MZDT110400A029A | V3 | 0.00 | 0.00 | 0.00 |
| MZDT110400A030A | V3 | 0.00 | 0.00 | 0.00 |
| MZDT110400A034A | V3 | 0.00 | 0.00 | 0.00 |
| MZDT110400A038A | V3 | 0.00 | 0.00 | 0.00 |
| MZDT110400A042A | V3 | 0.00 | 0.00 | 0.00 |
| MZDT110400A044A | V3 | 0.00 | 0.00 | 0.00 |
| MZDT110400A052A | V3 | 0.00 | 0.00 | 0.00 |
| MZDT110400A056A | V3 | 0.00 | 0.00 | 0.00 |
| MZDT110400A060A | V3 | 134.92 | 20.58 | 0.00 |
| MZDT110400A061A | V3 | 995.79 | 35.64 | 0.00 |
| MZDT110400A066A | V3 | 73.51 | 8.48 | 0.00 |
| MZDT110400A068A | V3 | 0.00 | 10.11 | 0.00 |
| MZDT110400A069A | V3 | 25.68 | 1.21 | 0.00 |
| MZDT110400A070A | V3 | 137.68 | 11.13 | 0.00 |
| MZDT110400A071A | V3 | 0.00 | 0.00 | 0.00 |
| MZDT110400B002A | V3 | 753.60 | 28.66 | 0.00 |
| MZDT110400B014A | V3 | 10.60 | 0.73 | 0.00 |
| MZDT110400B022A | V3 | 68.29 | 7.98 | 0.00 |
| MZDT110400B033A | V3 | 21.03 | 0.88 | 0.00 |
| MZDT110400B053A | V3 | 25.14 | 6.00 | 0.00 |
| MZDT110400A006A | V3 | 58.04 | 12.09 | 2.09 |
| MZDT110400A022A | V3 | 3355.38 | 215.42 | 2.30 |
| MZDT110400A007A | V3 | 81.23 | 7.25 | 2.31 |
| MZDT110400A041A | V3 | 18.95 | 4.04 | 2.95 |
| MZDT110400B019A | V3 | 6.39 | 1.05 | 3.57 |
| MZDT110400B052A | V3 | 71.77 | 3.00 | 5.07 |
| MZDT110400A032A | V3 | 57.84 | 5.31 | 5.39 |
| MZDT110400A048A | V3 | 228.05 | 15.36 | 5.66 |
| MZDT110400B003A | V3 | 21.13 | 1.73 | 5.71 |
| MZDT110400A003A | V3 | 306.16 | 124.48 | 6.14 |
| MZDT110400A063A | V3 | 33.32 | 1.98 | 6.17 |
| MZDT110400A049A | V3 | 5134.55 | 698.96 | 6.98 |
| MZDT110400A050A | V3 | 257.05 | 28.80 | 7.26 |
| MZDT110400B028A | V3 | 144.92 | 22.69 | 7.99 |
| MZDT110400A064A | V3 | 4870.13 | 175.26 | 8.35 |
| MZDT110400B055A | V3 | 55.11 | 5.65 | 8.36 |
| MZDT110400B024A | V3 | 78.24 | 7.06 | 9.66 |
| MZDT110400B030A | V3 | 30.45 | 10.53 | 9.84 |
| MZDT110400A055A | V3 | 341.12 | 16.40 | 11.54 |
| MZDT110400B049A | V3 | 47.96 | 18.28 | 11.64 |
| MZDT110400B054A | V3 | 32.96 | 5.40 | 12.24 |
| MZDT110400A026A | V3 | 610.17 | 36.03 | 13.06 |
| MZDT110400B016A | V3 | 490.50 | 13.45 | 13.92 |
| MZDT110400A067A | V3 | 322.74 | 33.26 | 14.68 |
| MZDT110400A062A | V3 | 6187.80 | 253.87 | 14.76 |
| MZDT110400B034A | V3 | 36.21 | 5.50 | 14.87 |
| MZDT110400B043A | V3 | 240.95 | 36.23 | 15.13 |
| MZDT110400B026A | V3 | 31.26 | 9.16 | 15.64 |
| MZDT110400B008A | V3 | 64.48 | 6.87 | 16.24 |
| MZDT110400B004A | V3 | 115.08 | 15.11 | 16.59 |
| MZDT110400B009A | V3 | 483.04 | 91.63 | 17.37 |
| MZDT110400B029A | V3 | 135.49 | 54.50 | 18.17 |
| MZDT110400B047A | V3 | 93.70 | 14.37 | 19.05 |
| MZDT110400B038A | V3 | 179.19 | 33.96 | 19.31 |
| MZDT110400A057A | V3 | 260.79 | 2.77 | 19.84 |
| MZDT110400A025A | V3 | 553.16 | 48.83 | 20.19 |
| MZDT110400B050A | V3 | 97.28 | 7.88 | 21.02 |
| MZDT110400B059A | V3 | 37.14 | 5.79 | 21.21 |
| MZDT110400B061A | V3 | 68.99 | 8.34 | 24.05 |
| MZDT110400B037A | V3 | 299.56 | 28.16 | 29.57 |
| MZDT110400B057A | V3 | 43.90 | 3.65 | 31.10 |
| MZDT110400A054A | V3 | 849.31 | 72.84 | 31.63 |
| MZDT110400A043A | V3 | 377.25 | 23.99 | 33.82 |
| MZDT110400A014A | V3 | 1099.78 | 38.27 | 33.85 |
| MZDT110400A024A | V3 | 1359.34 | 125.31 | 35.87 |
| MZDT110400B058A | V3 | 7932.19 | 1319.21 | 44.66 |
| MZDT110400A037A | V3 | 123.29 | 8.10 | 51.29 |
| MZDT110400A059A | V3 | 5678.81 | 221.06 | 51.90 |
| MZDT110400A053A | V3 | 548.86 | 31.92 | 60.48 |
| MZDT110400B041A | V3 | 558.96 | 30.83 | 60.58 |
| MZDT110400B036A | V3 | 236.52 | 9.99 | 69.78 |
| MZDT110400B046A | V3 | 522.21 | 40.71 | 70.59 |
| MZDT110400A001A | V3 | 1901.35 | 258.93 | 74.39 |
| MZDT110400B035A | V3 | 4041.00 | 195.39 | 78.64 |
| MZDT110400A011A | V3 | 717.66 | 60.06 | 103.56 |
| MZDT110400A045A | V3 | 1758.04 | 58.66 | 141.50 |

TABLE 18
Summary of GUS expression in T0 tassel leaf
sampled at R1 in select 19711 plants

| | |
|---|---|
| Sam- | ELISA (ng GUS/ |
| pling | mg total protein) |

| Event ID | Stage | Mean | St Dev |
|---|---|---|---|
| MZDT110400A013A | R1 | 0.00 | NA |
| MZDT110400A061A | R1 | 0.00 | NA |
| MZDT110400A070A | R1 | 0.00 | NA |
| MZDT110400A018A | R1 | 0.00 | NA |
| MZDT110400A022A | R1 | 3.14 | NA |
| MZDT110400A007A | R1 | 4.22 | NA |
| MZDT110400A006A | R1 | 5.61 | NA |
| MZDT110400A057A | R1 | 5.86 | NA |
| MZDT110400A032A | R1 | 11.76 | NA |
| MZDT110400A048A | R1 | 12.00 | NA |
| MZDT110400A067A | R1 | 12.80 | NA |
| MZDT110400A037A | R1 | 15.50 | NA |

TABLE 19
Summary of GUS and PMI expression in T1 leaf sampled at V3 in select 19711 plants

| | | | | | | | ELISA (ng | |
|---|---|---|---|---|---|---|---|---|
| | | | GUS qRT-PCR | | PMI qRT-PCR | | GUS/mg | |
| | Plant | Sampling | Relative | | Relative | | total protein) | |
| Event ID | Number | Stage | Mean | St Dev | Mean | St Dev | Mean | St Dev |
| MZDT110400A061A | 1 | V3 | 0 | 0 | 0 | 0 | ND | 1.66 |
| MZDT110400A061A | 2 | V3 | 27.06 | 2.09 | 33535.54 | 1369.45 | ND | 2.07 |
| MZDT110400A061A | 3 | V3 | 46.23 | 5.21 | 37512.41 | 4516.28 | 0.35 | 2.34 |
| MZDT110400A061A | 4 | V3 | 46.45 | 3.76 | 30576.22 | 4187.2 | 1.8 | 2 |
| MZDT110400A061A | 5 | V3 | 47.41 | 2.55 | 20808.77 | 3180.01 | 0.72 | 1.55 |
| MZDT110400A037A | 1 | V3 | 132.07 | 4.25 | 5079.94 | 389.78 | 10.25 | 2.09 |
| MZDT110400A037A | 2 | V3 | 132.94 | 6.19 | 13472.09 | 1868.36 | 7.46 | 2.61 |
| MZDT110400A037A | 3 | V3 | 164.68 | 12.02 | 17796.34 | 1916.07 | 15.37 | 2.07 |
| MZDT110400A037A | 4 | V3 | 0 | 0 | 0 | 0 | ND | 1.87 |
| MZDT110400A037A | 5 | V3 | 225.3 | 9.55 | 15083.25 | 1331.19 | 11.59 | 2.32 |
| MZDT110400A037A | 6 | V3 | 55.21 | 13.77 | 11499.13 | 920.77 | 15.07 | 2.1 |
| MZDT110400A037A | 7 | V3 | 198.94 | 9.09 | 12746.25 | 1075.42 | 11.83 | 2.88 |
| MZDT110400B054A | 1 | V3 | 89.65 | 2.62 | 25140.89 | 3212.89 | 6.66 | nd |
| MZDT110400B054A | 2 | V3 | 232.55 | 2.94 | 30205.97 | 4412.13 | 11.7 | 2.54 |
| MZDT110400B054A | 3 | V3 | 0 | 0 | 0 | 0 | ND | ND |
| MZDT110400B054A | 4 | V3 | 101.47 | 7.1 | 14093.5 | 258.162 | 5.79 | 2.43 |
| MZDT110400B054A | 5 | V3 | 69.65 | 2.86 | 8871.647 | 1450.18 | 6.41 | 1.28 |
| MZDT110400B054A | 6 | V3 | 174.16 | 16.867 | 11599.43 | 2318.17 | 5.62 | 1.6 |
| MZDT110400B054A | 7 | V3 | 102.56 | 1.17 | 10624.31 | 638.98 | 4.51 | ND |
| MZDT110400A061A | 1 | | | | | | | |

TABLE 20
Tassel leaf GUS ELISA at R1 stage

| | | |
|---|---|---|
| | Sam- | ELISA (ng GUS/ |
| Plant | pling | mg Total Protein) |

| Event ID | Number | Stage | Mean | St Dev |
|---|---|---|---|---|
| MZDT110400A061A | 1 | R1 | ND | NA |
| MZDT110400A061A | 2 | R1 | ND | NA |
| MZDT110400A061A | 3 | R1 | 0.35 | NA |
| MZDT110400A061A | 4 | R1 | 1.8 | NA |
| MZDT110400A061A | 5 | R1 | 0.72 | NA |
| MZDT110400A037A | 1 | R1 | 10.25 | NA |
| MZDT110400A037A | 2 | R1 | 7.46 | NA |
| MZDT110400A037A | 3 | R1 | 15.37 | NA |
| MZDT110400A037A | 4 | R1 | ND | NA |
| MZDT110400A037A | 5 | R1 | 11.59 | NA |
| MZDT110400A037A | 6 | R1 | 15.07 | NA |
| MZDT110400A037A | 7 | R1 | 11.83 | NA |
| MZDT110400B054A | 1 | R1 | 6.66 | NA |
| MZDT110400B054A | 2 | R1 | 11.7 | NA |
| MZDT110400B054A | 3 | R1 | ND | NA |
| MZDT110400B054A | 4 | R1 | 5.79 | NA |
| MZDT110400B054A | 5 | R1 | 6.41 | NA |
| MZDT110400B054A | 6 | R1 | 5.62 | NA |
| MZDT110400B054A | 7 | R1 | 4.51 | NA |

In summary, the 19711 expression cassette (SEQ ID NO:28) produces no GUS protein accumulation in leaf guard cells. The quantitative evidence suggests that the original version of this promoter, which works in Arabidopsis and tobacco, does not function in maize. Expression in other tissues was investigated in different T1 tissues such as husk, cob, stem, root, tassel and kernel. The GUS protein was detected in stem, cob and kernels.

The foregoing is illustrative of the present invention, and is not to be construed as limiting thereof. The invention is defined by the following claims, with equivalents of the claims to be included therein.

1.-2. (canceled)

3. A recombinant polynucleotide comprising a nucleic acid having at least 90% identity over the entire length of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, or SEQ ID NO:27.

4. A recombinant polynucleotide comprising a nucleic acid selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, and SEQ ID NO:27.

5. An expression cassette comprising a recombinant polynucleotide selected from the group consisting of the recombinant polynucleotide of claim 3, and the recombinant polynucleotide of claim 4.

6.-13. (canceled)

14. A cell comprising a recombinant polynucleotide selected from the group consisting of the recombinant polynucleotide of claim 3, and the recombinant polynucleotide of claim 4.

15. The cell of claim 14, wherein the cell is a plant cell or a bacterial cell.

16. A plant or plant part comprising a recombinant polynucleotide selected from the group consisting of the recombinant polynucleotide of claim 3, and the recombinant polynucleotide of claim 4.

17. A method of expressing a polynucleotide of interest in a guard cell of a plant, comprising
introducing into a plant a recombinant polynucleotide selected from the group consisting of the recombinant polynucleotide of claim 3, and the recombinant polynucleotide of claim 4; and
expressing the polynucleotide of interest.

18. The method of claim 17, wherein the expression of the polynucleotide of interest in the guard cell is specific or preferential.

19. The method of claim 17, wherein the polynucleotide of interest encodes an abscisic acid receptor or a modified abscisic acid receptor.

20. (canceled)

21. A plant and/or plant part produced by the method of claim 17.

22. (canceled)

23. A seed comprising a recombinant polynucleotide selected from the group consisting of the recombinant polynucleotide of claim 3, and the recombinant polynucleotide of claim 4.

24. A crop comprising a plurality of plants comprising a recombinant polynucleotide selected from the group consisting, the recombinant polynucleotide of claim 3, and the recombinant polynucleotide of claim 4 or a progeny thereof, wherein said progeny is a transgenic plant, planted together in an agricultural field.

25. A product produced from the plant of claim 21.

26. (canceled)

27. A recombinant polynucleotide comprising a nucleic acid selected from the group consisting of the nucleic acid of SEQ ID NO:3, SEQ ID NO:4, SEQ ID NO:5, SEQ ID NO:6, and SEQ ID NO:7.


SEQUENCE LISTING (30 sequences)

SEQ ID NO: 1 - DNA, 842 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcag

SEQ ID NO: 2 - DNA, 91 bp, Zea mays
gtaaactgttaaaaccattttcaagactaccttttctctatttcagacaaaccaaagtaaaacaatgaaaaatctctctggtctttcatag

SEQ ID NO: 3 - DNA, 1010 bp, Zea mays
gtacgccgctcgtcctccccccccccccctctctaccttctctagatcggcgttccggtccaaggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 4 - DNA, 1648 bp, Zea mays
gtgcgtcttgacggccggattcggctccccctcgcgcgccctgctccccaaaggcgccgcctttgctctgatccgtcgctctgcgcgctcgtggctgcttgtgatctctcctctgcggggcggtttcccggccggttactatcgcgggggggcggaactgggtcgctgggccgtggttcttggtgtcccggagcgccgaatctggcgcccctggaggaggagcgtgggagggtctcccgcgccacgcccgcggccggtcgatccggaattcgttcagttcgtctgcttaggctgtcttctttttcaactccttgcgaaattcgtagcatctggagcttggggaaagttccgtcgtgtgatctaatcccgtgctttttttccttcggtttgttcgagggaaacgttcttctgcttcggtattttggtcgtctgttgcaagcaagcaatttctggttttcaggtgtccgggaagcagcgttactgtccccatcgccgtcggggattaggggaaaattgcgccttttcctttctttttgcaagctgtgtcccactccatactggcttcgattggattggattggatagatagctcgtccgtggcgtggcacagtgtagcattgatagcccagcggagtgatgcttcgctgctgaagcaatcgcaggtttggcggagctaaggaaagagctgtagcgagcacgatgttagccttttgaattggcctggccctgttccatgagctggcgtgtgtgagaatggcgtgagtgtgagctgtgggcacgaggggaaaggtgagaccccccttggtgctggactgctggtgctgtggggggagcggcagtgcatgagatgcatgtgatgcggcattaaattttagtgtagccacgattgagatgaatctataggtgaagcgaatgaagtgactaatgtggtttcatcagactcaggaatttgttagtattcgtccattgccaaagctgactggtggatgtttaatctagtcgtagcaatagatttccaaccaatgtgtgtaattattgtctcgagctaaggtggcaagaaacttatggcgtattaagaattgtccaaggttttccatgcagaatgagctgttctttgatcagcttcgtagcaactggtgacttgagtagttagtaccttcctgccacgtatattttaatctacctggggtggttgggaaacaataaaggacaaaggattgcataaatcgtttgcatcatgtgggtcaaatttggttgccgtgcacctgtgttagtctcacatttggtatgtgaagataatgaatgcggaactgcagacaggggagaaggggtagagagaatttgtacatggtcttgtttggtgcaatgagtgcatgacccttgtaaccaggttcctggatcaatgaggtcgaagaacggaaacatggtacgtggtaagatactttaaacgagattttaacactacgtggacgaaattgttcccatgttcccggaacgaggaacgtggcggtttctaagtgcatgacattgatatcggttacttaatggaattggttgcttgctttctaagtgatttcgtttcctttgttcgtttttctgaccggtgctatattgctgccgcctctaataaaaatgtgtttgatttcag

SEQ ID NO: 5 - DNA, 1969 bp, Zea mays
gtgagtcggtgcgcgacccctcccctcctcaggtcgattcccactcccctccacctgcggattcccatggcgcaaggacgtggttcgctgatccgcttgccggatctacgcccttcgctccatccggttaattgtagccttttgtacccaggccgcgcggcgctgccgtaggttcgataccctgctgtacttgtcactgggatagattgcggtggttttacgcctcattggatattcccaaatggttatggaccctactagaggcctgcgattaatagcgactaatcgtccgattagtttaggaacgatcagcctggctggctggattgaaatttcggcccagtggcccacttcacttgcccagcggcccagcccagcttcccttttcccgattcccaccacgtgagcagaagctgcacaaccaagaggcaagagccaacagagcgcagaagctgcacaaccaagaggtaagagccaaccgagcaacgaacgcccaggcacacacgagacacgacacgaaccaggcaaccagctgccgagggaggaaccctagcggaacactgtgcaggtgacggcggcgcgctaccggtgacgggggatacggcggcatcgtccccctcggcccctcctccatccccggcgcggcggcgccagcagccggcctgcaggtacggtctccagtctccagaatccagattcagagcttgtccctgttttctggtgtgctctgttgtgctgtagcctgtaggtacgggcgtttggctgttggcctgttgctgcccatgtccatgtttgttggttttcagttttacttagatgctgaaagcctgaaaccttccagtgattatttgcttcatgttgtagtatggctgttggcctgttgctgcccgtgtccatgggcatatcacgtatggctgtttgccttgtcgaaagcctgaacctgaagccatgtccatgggcgtgtagttatatatatgaaggttgatcggttcctatagccctggacgatcaggatgtctgattgaccaatcagagcccggttaatcgggctggtcatcgactaatcgtgattagtcggctgatcgcccctctggcgatcaggagcgatcagccgatctgaaaacattgcgatctccctatgccgatctccctccggctctgaaatttaatactgcagcaaggtccaaactcagcctgggtgattccaactcagcttggaatcacaatttagcgtgggtgattccaaactcgacctgggtggttaatcgctttaccaaccatccgtgagtacaataactacttgcaagacaggtataattttgaagtcacttggttcagacttaaagttgctttgcagtaatcagtatgcagttgggatggcccatttcatctcaacatcattgttattccaatagcggcaattttgcatgatcagtctgctgagtggcgacttttcaaactgtgacggtcatcagtatccggaaggggaggatggcgtcggcgaagtaagaacagccggcggcgctgtgccaagctgtgctcagctcaaagaaatagccagtctcggtctccagttctcgtctccagtcaaggtgataaaaaaagacatgggtagaacggtccgtgaggcttactggcctgaggaaaggaaacacaaaacatttaaaatgaagataaggaccaaaaacacctagaatgaaaaatccgtgagccatggtgtcaatttctaaaaggatgatgaattggatcctttatgttgagaggcagccgaattgagacgtgtatactgaaatttctcccttgagcatcgatgtacataaaaaatgctttttgtatgatttagtgttacgtcgagatcatggatgcatcacttttctgctgctccttgggtcctcatgtaattgggcatctactgtgtggcaacatttttgtatgactgaatggtttccccacctctttatctctgaag

SEQ ID NO: 6 - DNA, 620 bp, Zea mays
gtatgccctctcctcccctttccttttcttatgttccttccccgctgtgcgacacccagcacctcaaaccctaacaatttgtatccggatctagatctgactcgttcacaatgcatatgcgttgtacacgctacccatctcctatgctccttcccttacatgcgtaatttcgatcttcctcgcagcgccggcgctccatcttccgctccctcccttactcgcgctcggcacacaggcgacggcggcggcgctcaggatcgggctatcaccgacggggtccggcttctccgcccgcctctcgccactcgtcctcgcctccggtcctccgcacaccggccggtgatcctgcgtgcgtgcctctctctctcctcccgcctctggtggatcgtcaacggcgcccggcctgagggcagcgggagccagcagccgctgctccgccaagagggtcgcgcgccgctttgggaacgcttatgctccaaagctaagcgttacccgtcctcttagcgctccagcgccctgaagcgtgcgcttagtgccgctttctagaaccctgtgtggcataaacaaccagctgaagtggaactgtttctgagcacccattttttgttttttcattgcag

SEQ ID NO: 7 - DNA, 792 bp, Zea mays
gtacgcccatcctctgctgtactccccccgttcctgctgtgcgaaaccgagcacccaaaccctagcctaagctattcgcatcgcaaactctattgagcgacggatcgcgaaacgcgcgccgccgcattcggacgagacctcgtggcccgtaccttcctactctaccgtcgtcgccggagacgagctcaccggttagggtttcgggattaggggcttggggcttggattcccgggggactccatacccggtggccttagaaggggaagggggcccagcggtggtggtggttcgactgtcagcgagcgagacggcggggcaccgccgatcgggcgtcgctggacatttgattgagctgggcaggcgaaggcgtggagcttgctcttcgattgggattagaggagggcggaggtggtatggtgggcggctcggctggctcaatggagccgctggccggtggtggggcaggcgagatcacgctctctcatagagcgtaatgggttgcgtaacacactcctgtttgtatttggatccgatctaataattaaagtttatttacgtggtgcacatgtagaattttcccatgcccactgctaacttggattggatttttgccaaaaaaacgggtgtgcacccaatcacttatagtgaaaccgttgtgtgtttgatgtgcttctaaccaagtacaaatccagtgaaaacacacctaccattcaacaatccacattttggttgcaattatcagcattctagaaaggtgcatgtgcccattgatacacttgctattggtgcag

SEQ ID NO: 8 - DNA, 602 bp, Zea mays
gtacgtgctcgcgtccgcgctctgcttttgctgctgcttccgaggtctgcgtgcgtgattcccgctgcctgcttcgcgcccggtggccgtgtcgtcgcctgcgggctgcggcttgcgagcgcgggaatctggtgcctccgggtcggcatggccccccgcgttaggccgccttgctcgattgcggcgtgggatttcgccccgcccgcctagcggtagcggcggggtgggtggaggcctggagccgtgggggtggttggctgccgccttctctggcccatttctcagagaaccccgcccgcacccggtagcttcctgcttcccgtagcattttccctcccatttcacgacaacttgctgcgtgcgccgctctgggtgggacctctgtgccttcctagttccttccttctatatgccgtgccgtgcgtccgtgggattggaacataagggcgaggcaggcagggcagtctttctgccgctattgctctttattatggcgagtggattttatggtagcccgcggcgttgcgattgcttctttcagcgtaagaataagatagattccgttctcaattggcgcttctttgggatggctctgtctgtag

SEQ ID NO: 9 - DNA, 12 bp, Arabidopsis thaliana
gtacttgcagag

SEQ ID NO: 10 - DNA, 933 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtaaactgttaaaaccattttcaagactaccttttctctatttcagacaaaccaaagtaaaacaatgaaaaatctctctggtctttcatag

SEQ ID NO: 11 - DNA, 1852 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtacgccgctcgtcctccccccccccccctctctaccttctctagatcggcgttccggtccaaggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 12 - DNA, 2490 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtgcgtcttgacggccggattcggctccccctcgcgcgccctgctccccaaaggcgccgcctttgctctgatccgtcgctctgcgcgctcgtggctgcttgtgatctctcctctgcggggcggtttcccggccggttactatcgcgggggggcggaactgggtcgctgggccgtggttcttggtgtcccggagcgccgaatctggcgcccctggaggaggagcgtgggagggtctcccgcgccacgcccgcggccggtcgatccggaattcgttcagttcgtctgcttaggctgtcttctttttcaactccttgcgaaattcgtagcatctggagcttggggaaagttccgtcgtgtgatctaatcccgtgctttttttccttcggtttgttcgagggaaacgttcttctgcttcggtattttggtcgtctgttgcaagcaagcaatttctggttttcaggtgtccgggaagcagcgttactgtccccatcgccgtcggggattaggggaaaattgcgccttttcctttctttttgcaagctgtgtcccactccatactggcttcgattggattggattggatagatagctcgtccgtggcgtggcacagtgtagcattgatagcccagcggagtgatgcttcgctgctgaagcaatcgcaggtttggcggagctaaggaaagagctgtagcgagcacgatgttagccttttgaattggcctggccctgttccatgagctggcgtgtgtgagaatggcgtgagtgtgagctgtgggcacgaggggaaaggtgagaccccccttggtgctggactgctggtgctgtggggggagcggcagtgcatgagatgcatgtgatgcggcattaaattttagtgtagccacgattgagatgaatctataggtgaagcgaatgaagtgactaatgtggtttcatcagactcaggaatttgttagtattcgtccattgccaaagctgactggtggatgtttaatctagtcgtagcaatagatttccaaccaatgtgtgtaattattgtctcgagctaaggtggcaagaaacttatggcgtattaagaattgtccaaggttttccatgcagaatgagctgttctttgatcagcttcgtagcaactggtgacttgagtagttagtaccttcctgccacgtatattttaatctacctggggtggttgggaaacaataaaggacaaaggattgcataaatcgtttgcatcatgtgggtcaaatttggttgccgtgcacctgtgttagtctcacatttggtatgtgaagataatgaatgcggaactgcagacaggggagaaggggtagagagaatttgtacatggtcttgtttggtgcaatgagtgcatgacccttgtaaccaggttcctggatcaatgaggtcgaagaacggaaacatggtacgtggtaagatactttaaacgagattttaacactacgtggacgaaattgttcccatgttcccggaacgaggaacgtggcggtttctaagtgcatgacattgatatcggttacttaatggaattggttgcttgctttctaagtgatttcgtttcctttgttcgtttttctgaccggtgctatattgctgccgcctctaataaaaatgtgtttgatttcag

SEQ ID NO: 13 - DNA, 2811 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtgagtcggtgcgcgacccctcccctcctcaggtcgattcccactcccctccacctgcggattcccatggcgcaaggacgtggttcgctgatccgcttgccggatctacgcccttcgctccatccggttaattgtagccttttgtacccaggccgcgcggcgctgccgtaggttcgataccctgctgtacttgtcactgggatagattgcggtggttttacgcctcattggatattcccaaatggttatggaccctactagaggcctgcgattaatagcgactaatcgtccgattagtttaggaacgatcagcctggctggctggattgaaatttcggcccagtggcccacttcacttgcccagcggcccagcccagcttcccttttcccgattcccaccacgtgagcagaagctgcacaaccaagaggcaagagccaacagagcgcagaagctgcacaaccaagaggtaagagccaaccgagcaacgaacgcccaggcacacacgagacacgacacgaaccaggcaaccagctgccgagggaggaaccctagcggaacactgtgcaggtgacggcggcgcgctaccggtgacgggggatacggcggcatcgtccccctcggcccctcctccatccccggcgcggcggcgccagcagccggcctgcaggtacggtctccagtctccagaatccagattcagagcttgtccctgttttctggtgtgctctgttgtgctgtagcctgtaggtacgggcgtttggctgttggcctgttgctgcccatgtccatgtttgttggttttcagttttacttagatgctgaaagcctgaaaccttccagtgattatttgcttcatgttgtagtatggctgttggcctgttgctgcccgtgtccatgggcatatcacgtatggctgtttgccttgtcgaaagcctgaacctgaagccatgtccatgggcgtgtagttatatatatgaaggttgatcggttcctatagccctggacgatcaggatgtctgattgaccaatcagagcccggttaatcgggctggtcatcgactaatcgtgattagtcggctgatcgcccctctggcgatcaggagcgatcagccgatctgaaaacattgcgatctccctatgccgatctccctccggctctgaaatttaatactgcagcaaggtccaaactcagcctgggtgattccaactcagcttggaatcacaatttagcgtgggtgattccaaactcgacctgggtggttaatcgctttaccaaccatccgtgagtacaataactacttgcaagacaggtataattttgaagtcacttggttcagacttaaagttgctttgcagtaatcagtatgcagttgggatggcccatttcatctcaacatcattgttattccaatagcggcaattttgcatgatcagtctgctgagtggcgacttttcaaactgtgacggtcatcagtatccggaaggggaggatggcgtcggcgaagtaagaacagccggcggcgctgtgccaagctgtgctcagctcaaagaaatagccagtctcggtctccagttctcgtctccagtcaaggtgataaaaaaagacatgggtagaacggtccgtgaggcttactggcctgaggaaaggaaacacaaaacatttaaaatgaagataaggaccaaaaacacctagaatgaaaaatccgtgagccatggtgtcaatttctaaaaggatgatgaattggatcctttatgttgagaggcagccgaattgagacgtgtatactgaaatttctcccttgagcatcgatgtacataaaaaatgctttttgtatgatttagtgttacgtcgagatcatggatgcatcacttttctgctgctccttgggtcctcatgtaattgggcatctactgtgtggcaacatttttgtatgactgaatggtttccccacctctttatctctgaag

SEQ ID NO: 14 - DNA, 1462 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtatgccctctcctcccctttccttttcttatgttccttccccgctgtgcgacacccagcacctcaaaccctaacaatttgtatccggatctagatctgactcgttcacaatgcatatgcgttgtacacgctacccatctcctatgctccttcccttacatgcgtaatttcgatcttcctcgcagcgccggcgctccatcttccgctccctcccttactcgcgctcggcacacaggcgacggcggcggcgctcaggatcgggctatcaccgacggggtccggcttctccgcccgcctctcgccactcgtcctcgcctccggtcctccgcacaccggccggtgatcctgcgtgcgtgcctctctctctcctcccgcctctggtggatcgtcaacggcgcccggcctgagggcagcgggagccagcagccgctgctccgccaagagggtcgcgcgccgctttgggaacgcttatgctccaaagctaagcgttacccgtcctcttagcgctccagcgccctgaagcgtgcgcttagtgccgctttctagaaccctgtgtggcataaacaaccagctgaagtggaactgtttctgagcacccattttttgttttttcattgcag

SEQ ID NO: 15 - DNA, 1634 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtacgcccatcctctgctgtactccccccgttcctgctgtgcgaaaccgagcacccaaaccctagcctaagctattcgcatcgcaaactctattgagcgacggatcgcgaaacgcgcgccgccgcattcggacgagacctcgtggcccgtaccttcctactctaccgtcgtcgccggagacgagctcaccggttagggtttcgggattaggggcttggggcttggattcccgggggactccatacccggtggccttagaaggggaagggggcccagcggtggtggtggttcgactgtcagcgagcgagacggcggggcaccgccgatcgggcgtcgctggacatttgattgagctgggcaggcgaaggcgtggagcttgctcttcgattgggattagaggagggcggaggtggtatggtgggcggctcggctggctcaatggagccgctggccggtggtggggcaggcgagatcacgctctctcatagagcgtaatgggttgcgtaacacactcctgtttgtatttggatccgatctaataattaaagtttatttacgtggtgcacatgtagaattttcccatgcccactgctaacttggattggatttttgccaaaaaaacgggtgtgcacccaatcacttatagtgaaaccgttgtgtgtttgatgtgcttctaaccaagtacaaatccagtgaaaacacacctaccattcaacaatccacattttggttgcaattatcagcattctagaaaggtgcatgtgcccattgatacacttgctattggtgcag

SEQ ID NO: 16 - DNA, 1444 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtacgtgctcgcgtccgcgctctgcttttgctgctgcttccgaggtctgcgtgcgtgattcccgctgcctgcttcgcgcccggtggccgtgtcgtcgcctgcgggctgcggcttgcgagcgcgggaatctggtgcctccgggtcggcatggccccccgcgttaggccgccttgctcgattgcggcgtgggatttcgccccgcccgcctagcggtagcggcggggtgggtggaggcctggagccgtgggggtggttggctgccgccttctctggcccatttctcagagaaccccgcccgcacccggtagcttcctgcttcccgtagcattttccctcccatttcacgacaacttgctgcgtgcgccgctctgggtgggacctctgtgccttcctagttccttccttctatatgccgtgccgtgcgtccgtgggattggaacataagggcgaggcaggcagggcagtctttctgccgctattgctctttattatggcgagtggattttatggtagcccgcggcgttgcgattgcttctttcagcgtaagaataagatagattccgttctcaattggcgcttctttgggatggctctgtctgtag

SEQ ID NO: 17 - DNA, 945 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtaaactgttaaaaccattttcaagactaccttttctctatttcagacaaaccaaagtaaaacaatgaaaaatctctctggtctttcataggtacttgcagag

SEQ ID NO: 18 - DNA, 1864 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtacgccgctcgtcctccccccccccccctctctaccttctctagatcggcgttccggtccaaggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtacttgcagag

SEQ ID NO: 19 - DNA, 2502 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtgcgtcttgacggccggattcggctccccctcgcgcgccctgctccccaaaggcgccgcctttgctctgatccgtcgctctgcgcgctcgtggctgcttgtgatctctcctctgcggggcggtttcccggccggttactatcgcgggggggcggaactgggtcgctgggccgtggttcttggtgtcccggagcgccgaatctggcgcccctggaggaggagcgtgggagggtctcccgcgccacgcccgcggccggtcgatccggaattcgttcagttcgtctgcttaggctgtcttctttttcaactccttgcgaaattcgtagcatctggagcttggggaaagttccgtcgtgtgatctaatcccgtgctttttttccttcggtttgttcgagggaaacgttcttctgcttcggtattttggtcgtctgttgcaagcaagcaatttctggttttcaggtgtccgggaagcagcgttactgtccccatcgccgtcggggattaggggaaaattgcgccttttcctttctttttgcaagctgtgtcccactccatactggcttcgattggattggattggatagatagctcgtccgtggcgtggcacagtgtagcattgatagcccagcggagtgatgcttcgctgctgaagcaatcgcaggtttggcggagctaaggaaagagctgtagcgagcacgatgttagccttttgaattggcctggccctgttccatgagctggcgtgtgtgagaatggcgtgagtgtgagctgtgggcacgaggggaaaggtgagaccccccttggtgctggactgctggtgctgtggggggagcggcagtgcatgagatgcatgtgatgcggcattaaattttagtgtagccacgattgagatgaatctataggtgaagcgaatgaagtgactaatgtggtttcatcagactcaggaatttgttagtattcgtccattgccaaagctgactggtggatgtttaatctagtcgtagcaatagatttccaaccaatgtgtgtaattattgtctcgagctaaggtggcaagaaacttatggcgtattaagaattgtccaaggttttccatgcagaatgagctgttctttgatcagcttcgtagcaactggtgacttgagtagttagtaccttcctgccacgtatattttaatctacctggggtggttgggaaacaataaaggacaaaggattgcataaatcgtttgcatcatgtgggtcaaatttggttgccgtgcacctgtgttagtctcacatttggtatgtgaagataatgaatgcggaactgcagacaggggagaaggggtagagagaatttgtacatggtcttgtttggtgcaatgagtgcatgacccttgtaaccaggttcctggatcaatgaggtcgaagaacggaaacatggtacgtggtaagatactttaaacgagattttaacactacgtggacgaaattgttcccatgttcccggaacgaggaacgtggcggtttctaagtgcatgacattgatatcggttacttaatggaattggttgcttgctttctaagtgatttcgtttcctttgttcgtttttctgaccggtgctatattgctgccgcctctaataaaaatgtgtttgatttcaggtacttgcagag

SEQ ID NO: 20 - DNA, 2823 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtgagtcggtgcgcgacccctcccctcctcaggtcgattcccactcccctccacctgcggattcccatggcgcaaggacgtggttcgctgatccgcttgccggatctacgcccttcgctccatccggttaattgtagccttttgtacccaggccgcgcggcgctgccgtaggttcgataccctgctgtacttgtcactgggatagattgcggtggttttacgcctcattggatattcccaaatggttatggaccctactagaggcctgcgattaatagcgactaatcgtccgattagtttaggaacgatcagcctggctggctggattgaaatttcggcccagtggcccacttcacttgcccagcggcccagcccagcttcccttttcccgattcccaccacgtgagcagaagctgcacaaccaagaggcaagagccaacagagcgcagaagctgcacaaccaagaggtaagagccaaccgagcaacgaacgcccaggcacacacgagacacgacacgaaccaggcaaccagctgccgagggaggaaccctagcggaacactgtgcaggtgacggcggcgcgctaccggtgacgggggatacggcggcatcgtccccctcggcccctcctccatccccggcgcggcggcgccagcagccggcctgcaggtacggtctccagtctccagaatccagattcagagcttgtccctgttttctggtgtgctctgttgtgctgtagcctgtaggtacgggcgtttggctgttggcctgttgctgcccatgtccatgtttgttggttttcagttttacttagatgctgaaagcctgaaaccttccagtgattatttgcttcatgttgtagtatggctgttggcctgttgctgcccgtgtccatgggcatatcacgtatggctgtttgccttgtcgaaagcctgaacctgaagccatgtccatgggcgtgtagttatatatatgaaggttgatcggttcctatagccctggacgatcaggatgtctgattgaccaatcagagcccggttaatcgggctggtcatcgactaatcgtgattagtcggctgatcgcccctctggcgatcaggagcgatcagccgatctgaaaacattgcgatctccctatgccgatctccctccggctctgaaatttaatactgcagcaaggtccaaactcagcctgggtgattccaactcagcttggaatcacaatttagcgtgggtgattccaaactcgacctgggtggttaatcgctttaccaaccatccgtgagtacaataactacttgcaagacaggtataattttgaagtcacttggttcagacttaaagttgctttgcagtaatcagtatgcagttgggatggcccatttcatctcaacatcattgttattccaatagcggcaattttgcatgatcagtctgctgagtggcgacttttcaaactgtgacggtcatcagtatccggaaggggaggatggcgtcggcgaagtaagaacagccggcggcgctgtgccaagctgtgctcagctcaaagaaatagccagtctcggtctccagttctcgtctccagtcaaggtgataaaaaaagacatgggtagaacggtccgtgaggcttactggcctgaggaaaggaaacacaaaacatttaaaatgaagataaggaccaaaaacacctagaatgaaaaatccgtgagccatggtgtcaatttctaaaaggatgatgaattggatcctttatgttgagaggcagccgaattgagacgtgtatactgaaatttctcccttgagcatcgatgtacataaaaaatgctttttgtatgatttagtgttacgtcgagatcatggatgcatcacttttctgctgctccttgggtcctcatgtaattgggcatctactgtgtggcaacatttttgtatgactgaatggtttccccacctctttatctctgaaggtacttgcagag

SEQ ID NO: 21 - DNA, 1474 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtatgccctctcctcccctttccttttcttatgttccttccccgctgtgcgacacccagcacctcaaaccctaacaatttgtatccggatctagatctgactcgttcacaatgcatatgcgttgtacacgctacccatctcctatgctccttcccttacatgcgtaatttcgatcttcctcgcagcgccggcgctccatcttccgctccctcccttactcgcgctcggcacacaggcgacggcggcggcgctcaggatcgggctatcaccgacggggtccggcttctccgcccgcctctcgccactcgtcctcgcctccggtcctccgcacaccggccggtgatcctgcgtgcgtgcctctctctctcctcccgcctctggtggatcgtcaacggcgcccggcctgagggcagcgggagccagcagccgctgctccgccaagagggtcgcgcgccgctttgggaacgcttatgctccaaagctaagcgttacccgtcctcttagcgctccagcgccctgaagcgtgcgcttagtgccgctttctagaaccctgtgtggcataaacaaccagctgaagtggaactgtttctgagcacccattttttgttttttcattgcaggtacttgcagag

SEQ ID NO: 22 - DNA, 1646 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtacgcccatcctctgctgtactccccccgttcctgctgtgcgaaaccgagcacccaaaccctagcctaagctattcgcatcgcaaactctattgagcgacggatcgcgaaacgcgcgccgccgcattcggacgagacctcgtggcccgtaccttcctactctaccgtcgtcgccggagacgagctcaccggttagggtttcgggattaggggcttggggcttggattcccgggggactccatacccggtggccttagaaggggaagggggcccagcggtggtggtggttcgactgtcagcgagcgagacggcggggcaccgccgatcgggcgtcgctggacatttgattgagctgggcaggcgaaggcgtggagcttgctcttcgattgggattagaggagggcggaggtggtatggtgggcggctcggctggctcaatggagccgctggccggtggtggggcaggcgagatcacgctctctcatagagcgtaatgggttgcgtaacacactcctgtttgtatttggatccgatctaataattaaagtttatttacgtggtgcacatgtagaattttcccatgcccactgctaacttggattggatttttgccaaaaaaacgggtgtgcacccaatcacttatagtgaaaccgttgtgtgtttgatgtgcttctaaccaagtacaaatccagtgaaaacacacctaccattcaacaatccacattttggttgcaattatcagcattctagaaaggtgcatgtgcccattgatacacttgctattggtgcaggtacttgcagag

SEQ ID NO: 23 - DNA, 1456 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtacgtgctcgcgtccgcgctctgcttttgctgctgcttccgaggtctgcgtgcgtgattcccgctgcctgcttcgcgcccggtggccgtgtcgtcgcctgcgggctgcggcttgcgagcgcgggaatctggtgcctccgggtcggcatggccccccgcgttaggccgccttgctcgattgcggcgtgggatttcgccccgcccgcctagcggtagcggcggggtgggtggaggcctggagccgtgggggtggttggctgccgccttctctggcccatttctcagagaaccccgcccgcacccggtagcttcctgcttcccgtagcattttccctcccatttcacgacaacttgctgcgtgcgccgctctgggtgggacctctgtgccttcctagttccttccttctatatgccgtgccgtgcgtccgtgggattggaacataagggcgaggcaggcagggcagtctttctgccgctattgctctttattatggcgagtggattttatggtagcccgcggcgttgcgattgcttctttcagcgtaagaataagatagattccgttctcaattggcgcttctttgggatggctctgtctgtaggtacttgcagag

SEQ ID NO: 24 - DNA, 1016 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtaaactgttaaaaccattttcaagactaccttttctctatttcagacaaaccaaagtaaaacaatgaaaaatctctctggtctttcataggtacttgcagagttgttttacaacaattaccaacaacaacaaacaacaaacaacattacaattagtatttacataaacgtaaa

SEQ ID NO: 25 - DNA, 949 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtaaactgttaaaaccattttcaagactaccttttctctatttcagacaaaccaaagtaaaacaatgaaaaatctctctggtctttcataggtacttgcagagtaaa

SEQ ID NO: 26 - DNA, 847 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtaaa

SEQ ID NO: 27 - DNA, 1868 bp, Artificial Sequence
  Artificial transcription regulating polynucleotide
tctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaataactaatcaagaaatttgaagaagttgattgtggtggcttttgttacgctgatcatctcttttcttctgctttctcaggtacgccgctcgtcctccccccccccccctctctaccttctctagatcggcgttccggtccaaggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtacttgcagagtaaa

SEQ ID NO: 28 - DNA, 1163 bp, Arabidopsis thaliana
atggttgcaacagagaggatgaatttataagttttcaacaccgcttttcttattagacggacaacaatctatagtggagtaaatttttatttttggtaaaatggttagtgaattcaaatatctaaattttgtgactcactaacattaacaaatatgcataagacataaaaaaaagaaagaataattcttatgaaacaagaaaaaaaacctatacaatcaatctttaggaattgacgatgtagaattgtagatgataaattttctcaaatatagatgggcctaatgaagggtgccgcttattggatctgacccattttgaggacattaatattttcattggttataagccttttaatcaaaattgtcattaaattgatgtctccctctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaatcactactcaagaaat

SEQ ID NO: 29 - DNA, 70 bp, Tobacco mosaic virus
tgttttacaacaattaccaacaacaacaaacaacaaacaacattacaattagtatttacataaacgtaaa

SEQ ID NO: 30 - DNA, 654 bp, Arabidopsis thaliana
aagacttctcatttctcaactatagtctcatcttctgattatgtttcttcttttgttatgttgcatgtgtgatgtgtgagcttattattatgttgattgttgacataattcaactatataatttgtatcgattccgaataataagatgagtgattttattggctattaagttttttttttttttttttgggcacaatggctattaagttttaaacatctgattttattggttacaaaaaacaacaaagtttcattttcatattaacacaaaatctccatacatattaccaaaccaaaaaaatacacaagggggagagagaccaacggttcttggttcagagtttgcatcttgtttgagccgtcaccgtttcttagacttaacagccacaacacctttataaagcttcacgcgatccttcaacgcatctcgccgaggccgagccaccttattgtttggatcaaacaacaaaacttcttcaaacgcattcatagccaaaggcaactccttcttcttctcataagcatccccaaggttgttccaagctgttacataaccgggttgcagcttcaccgcctattcaaactgagcaattcctttgtcaagtttatcctctcgtacataactcactccaagagcgttatagacctgtctca